(19) United States
(12) Patent Application Publication
(10) Pub. No.: US 2026/0325749 A1
(43) Pub. Date: Oct. 22, 2026

(54) Closed-System Infusion Adapter with High-Strength Connection to an Infusion Fluid Container

(71) Applicant: Becton, Dickinson and Company, Franklin Lakes, NJ (US)

(72) Inventors: Sanskriti Jain, Shahdara (IN); Praveen Nalawade, Bangaluru (IN); Mukilan Balamurugan, Theni (IN); Jeevan S. D., Puttur Talluk (IN); Edwin Jeyaseelan A., Madurai (IN); Sachin Chandrakant Gawali, Bangalore (IN); Saket Raj, Patna (IN); Aman Desai, Bengaluru (IN); Dennis Lionel A., Coimbatore (IN); Sri Harsha Sudhi Reddy, Hyderabad (IN); Jacob Daniel Crider, Sandy, UT (US); Manish Kumar, Bengaluru (IN); Mohammed Mehtab Khan, Bengaluru (IN); Amit Kumar, Bengaluru (IN); Ashish Mehta, Bangalore (IN); Rahul V .M., Mysore (IN); Srushti Umesh Deore, Nashik (IN); S. Monish Kumar, Chennai (IN); Pawan Kumar Yadav, Haryana (IN); Jayakumar Jayabal, Bangalore (IN); Deivakumaran Rajendran, Bengaluru (IN); Kowshika K., Tirupur (IN); Ajay Vijay Suryavanshi, Pune (IN); Amarendra Talagana, Andhra Pradesh (IN)

(21) Appl. No.: 19/184,447

(22) Filed: Apr. 21, 2025

(51) Int. Cl. A61M 5/162; A61J 1/20

(52) U.S. Cl. CPC A61M 5/162; A61J 1/201; A61J 1/2055

(57) ABSTRACT

A fluid transfer system is provided that includes an infusion fluid container containing an infusion fluid therein, an infusion adapter for connection with the infusion fluid container, and a securing mechanism for securing the infusion fluid container and infusion adapter together. The infusion fluid container includes a port having a fluid barrier member therein. The infusion adapter includes a connection spike having a main spike portion and a puncturing point configured to engage the port, with the port positioned about the main spike portion and the puncturing point when engaged therewith. The securing mechanism is positionable about the port and the main spike portion after the main spike portion has been engaged with the port, to secure the port and the main spike portion together. The securing mechanism restricts vertical movement between the port and the main spike portion to prevent dislodging of the main spike portion from the port.

### BACKGROUND OF THE INVENTION

### Field of the Invention

The present disclosure relates generally to an infusion adapter and, more particularly, to a high-strength connector for securing a closed-system infusion adapter to an infusion fluid container for a drug transfer procedure.

### Description of Related Art

Intravenous therapy applications allow patients to receive infusion and medication treatment. For example, therapy may include the administration of medications by IV using intravenous and subcutaneous or hypodermis routes, i.e., into the bloodstream and under the skin. Examples of medical treatments that intravenous therapy applications may provide to a patient include antibiotics, pain management medications, cancer treatments, and similar medications.

Medications may be packaged as “pre-filled” devices, wherein a syringe assembly is pre-filled with medication prior to being packaged and delivered to a patient. “Pre-filled” devices eliminate the need for a user to fill the device prior to injection.

Certain drugs or medications are preferably provided in powder or dry form (such as a lyophilized form) and require reconstitution prior to administration. Lyophilized drugs, for example, typically are supplied in a freeze-dried form that needs to be mixed with a diluent to reconstitute the substance into a form that is suitable for injection. In addition, drugs may be provided as multipart systems that require mixing prior to administration. For example, one or more liquid components, such as flowable slurries, and one or more dry components, such as powdered or granular components, may be provided in separate containers that require mixing prior to administration.

A patient may be provided with an intravenous system that includes intravenous tubing and a connector that is adapted to receive an injector and/or syringe assembly containing a required medication. In this manner, when a treatment is needed, a patient or a medical practitioner is able to connect a syringe assembly to the connector and then inject a medication intravenously into the patient via the injector and/or syringe assembly, the connector, and the intravenous tubing.

When performing infusion, it is often necessary to inject a drug or other medical substance into the infusion fluid inside an infusion bag or other infusion fluid container. This is often done by means of penetrating a septum or other fluid barrier of an injection port on the infusion fluid container, and this may be done by an infusion adapter having a spike connector thereon, with a syringe in-turn connected to the infusion adapter that is filled with the medical fluid in question. In use of such an infusion adapter, the spike connector is pushed into the injection port on the infusion fluid container, thereby mechanically and fluidly connecting the infusion adapter and the infusion fluid container. However, it is recognized that the design of existing infusion adapters is such that engagement between the spike connector and the injection port does not provide a secure engagement that prevents dislodging of the spike connector from the injection port. In the event that the spike connector is dislodged from the injection port, medication may leak from the infusion fluid container, which may lead to hazardous drug exposure and/or costly medication waste.

Accordingly, it is desirable to provide a means for securely engaging an infusion adapter with an infusion fluid container, in order to prevent dislodging of the spike connector of the adapter from the injection port and potential medication leakage from the infusion fluid container.

### SUMMARY OF THE INVENTION

In accordance with an embodiment of the present invention, a fluid transfer system includes an infusion fluid container containing an infusion fluid therein. The infusion fluid container includes a port having a fluid barrier member therein, and an infusion adapter for connection with the infusion fluid container. The infusion adapter includes a connection spike positioned at a first end of the infusion adapter, the connection spike having a main spike portion and a puncturing point configured to engage the port, with the port positioned about the main spike portion and the puncturing point when engaged therewith. The fluid transfer system also includes a securing mechanism positionable about the port and the main spike portion with the main spike portion engaged with the port, to secure the port and the main spike portion together. The securing mechanism restricts vertical movement between the port and the main spike portion to prevent dislodging of the main spike portion from the port.

In certain configurations, the securing mechanism includes a hinge clamp configured to apply a compression force against the engaged port and main spike portion to prevent dislodging of the main spike portion from the port. The hinge clamp may include a clamp member including a flexible leg assembly and an annular-shaped holder with slits formed therein to separate the holder into a pair of holder portions that may be selectively joined and separated, the holder positionable about the engaged port and main spike portion and configured to apply the compression force thereto. The hinge clamp may also include a slider translatable relative to the clamp member, with the slider applying an inward-directed force against the flexible leg assembly as the slider is moved from a first end of the flexible leg assembly toward a second end of the flexible leg assembly, thereby causing the pair of holder portions to join and apply the compression force to the engaged port and main spike portion.

In certain configurations, the flexible leg assembly includes a first leg and a second leg each including a plurality of teeth formed on an outward facing surface thereof, and wherein the slider includes a pair of tongues that engage with the plurality of teeth on the first and second legs, with the pair of tongues engaging with the plurality of teeth to prevent movement of the slider from the second end of the flexible leg assembly back toward the first end of the flexible leg assembly, so as to lock the annular-shaped holder in a closed configuration. The hinge clamp may further include a screw adjuster operable to actuate the slider. The screw adjuster may include a thumb screw inserted through a hole in the flexible leg assembly, with the thumb screw being rotatable to linearly advance the thumb screw relative to the flexible leg assembly. The hinge clamp may further include a slider connector coupling the thumb screw to the slider, such that linear movement of the thumb screw relative to the clamp member is transferred to slider, with the slider thus moving linearly relative to the clamp member responsive to rotation of the thumb screw.

In certain configurations, the hinge clamp includes a bi-stable hinge clamp including a clamp member including a first leg and a second leg joined together at a first end thereof via a connecting portion, an annular-shaped holder with slits formed therein to separate the holder into a pair of holder portions, with a respective holder portion joined to each of the first leg and the second leg, a shaft inserted through a hole in the connecting portion so as to be movable relative to the clamp member, the shaft including a button at a first end thereof, and a live hinge connecting the clamp member to the shaft, the live hinge comprising a plurality of hinge members that extend between the shaft and each of the first leg and the second leg, wherein the plurality of hinge members comprise flexible members that are actuatable from an extended configuration to a collapsed configuration responsive to movement of the shaft. With the shaft in a first position, the hinge members are in the extended configuration, so that the first leg and the second leg are in an open position, with the pair of holder portions thus separated from one another, and with the shaft in a second position, the hinge members are in the collapsed configuration, so that the first leg and the second leg are in a closed position, with the pair of holder portions moved into contact with each other to apply the compression force to the engaged port and main spike portion.

In certain configurations, the securing mechanism includes a sleeve clamp including a pair of flexible sleeve portions separated by a slit, with the slit being expandable upon a pressure being applied thereto, so as to enable the engaged port and spike portion to be pushed into a receptacle defined by the pair of flexible sleeve portions. The sleeve clamp may include a locking mechanism configured to lock the pair of flexible sleeve portions together.

In certain configurations, an inner surface of the pair of flexible sleeve portions includes a plurality of teeth spaced circumferentially thereabout, with each of the plurality of teeth extending radially inward and providing increased friction between the sleeve clamp and the port. The connection spike may include a spike collar formed about the main spike portion on an end thereof opposite the puncturing point, and/or wherein the port comprises a port collar formed thereon. The securing mechanism may include a hinge lock enclosure clamp including a pair of brackets coupled together in a hinged arrangement, with the hinge lock enclosure clamp enclosing a region where the port is engaged to the spike portion, and a locking mechanism provided on the pair of brackets and configured to lock the pair of brackets together.

In certain configurations, each of the pair of brackets includes a C-shaped channel defined therein that extends a length of the bracket, with opposing end walls of the bracket having semi-circular cut-out formed therein and a pair of C-shaped protrusions being provided on an inner surface of the bracket. Optionally, the hinge lock enclosure clamp is positioned about the engaged port and main spike portion, the spike collar and the port collar are retained between the pair of C-shaped protrusions, so as to restrict vertical movement between the port and the main spike portion.

In certain configurations, each of the pair of C-shaped protrusions includes a silicone pad thereon that forms a seal with the port and the main spike portion, to prevent leakage therefrom. At least one of the pair of brackets includes a rubber gasket arranged about a perimeter of the bracket, where the bracket is brought into engagement with the other bracket, with the rubber gasket seals an interior of the hinge lock enclosure clamp to prevent any leakage out therefrom.

In certain configurations, the hinge lock enclosure clamp includes an absorbing material included within the interior that is configured to absorb any leakage of fluid. Optionally, each of the pair of brackets includes a C-shaped channel defined therein that extends a length of the bracket, with opposing end walls of the bracket having semi-circular cut-out formed therein, and wherein each of the pair of brackets includes a C-shaped slot formed on an inner surface thereof configured to receive and retain the spike collar therein, and thereby restrict vertical movement between the port and the main spike portion. Optionally, these semi-circular cut-out includes an adhesive applied thereto that is configured to secure the hinge lock enclosure clamp to the insertion port. Further optionally, the locking mechanism may include one of a hinge pin on a first bracket of the pair of brackets and a second bracket of the pair of brackets, or a snap or tab on each of the first bracket and second bracket that are configured to engage each other.

In certain configurations, the securing mechanism includes a press-fit clamp including a first enclosure tab and a second enclosure tab that, when joined together, define a channel therein configured to receive the engaged port and spike portion, with the first enclosure tab including a plurality of pins thereon configured to engage a plurality of receptacles on the second enclosure tab to lock the press-fit clamp together. Each of the first and second enclosure tabs may include a semi-circular cut-out formed at each of opposing ends thereof, with one or more of the semi-circular cut-outs including a rib formed thereabout configured to engage an outer surface of the port when the press-fit clamp is positioned about the engaged port and spike portion, to increase friction between the press-fit clamp and the port.

In certain configurations, the securing mechanism includes a catch clip that includes an holder that defines an opening or receptacle configured to receive the engaged port and spike portion, the holder comprising an upper member and a lower member separated vertically by a gap, with the gap sized to receive the spike collar and port collar therein, and thereby restrict vertical movement between the port and the main spike portion. Optionally, the holder includes an annular-shaped holder having a slit formed therein that provides entry into the opening or receptacle, and wherein the catch clip includes finger grips positioned on opposing sides of the annular-shaped holder and extending radially outward therefrom. Responsive to application of a pinching force to the finger grips, the catch clip is caused to flex such that a width of the slit is increased and access to the opening or receptacle of holder is provided.

Optionally, the catch clip includes a semi-circular first and second holder portions that collectively form the holder, the first and second holder portions connected together at a pivot point and movable between an open position and closed position. The catch clip may also include first and second actuation arms provided on the first and second holder portions and actuatable by a user to move the first and second holder portions from the open position to the closed position, and a locking arm joined to the first holder portion and oriented such that it may be brought into engagement with the second holder portion when the catch clip is moved to the closed position. The locking arm may also include a plurality or serrations or teeth formed thereon that may engage serrations or teeth formed on an outer surface of the second holder portion to lock the catch clip may be locked in the closed position.

In certain configurations, the catch clip includes a first holder arm and a second holder arm that collectively form the holder, each of the first and second holder arms having a C-shaped construction and including a first end and a second end, and a connecting member coupled to the first and second holder arms at the first end. The second end of the first holder arm may be separated from the second end of the second holder arm by a gap, and the second end of the first holder arm and the second holder arm may each include a protrusions formed thereon, the protrusions on the first and second holder arms angled away from each other, so as to provide a funnel sloping toward the gap, with a size of the gap expanding upon a pushing of the catch clip onto the engaged port and main spike portion.

In certain configurations, the securing mechanism may include a catch clip including a clip portion comprising a first clip arm and a second clip arm that, when in a closed arrangement, provide a holder defining an opening or receptacle within which the engaged port and spike portion may be retained, each of the first clip arm and a second clip arm including a C-shaped portion and a finger grip portion. The catch clip may also include a spring coupling the first clip arm and the second clip arm and that biases the first clip arm and second clip arm in the closed arrangement, with the first clip arm and second clip arm moving to an open arrangement upon a pinching force being applied to the finger grip portions, and a locking mechanism via which the first clip arm and second clip arm may be locked together to secure the catch clamp in place about the engaged port and spike portion, and a claw portion comprising a first claw arm and a second claw arm each formed as a C-shaped arm. The first claw arm and the second claw arm may be joined to the first clip arm and the second clip arm, respectively, by support members, with movement of the first clip arm and the second clip arm causing a corresponding movement of the first claw arm and the second claw arm. The support members may space the claw arm and the second claw arm vertically apart from the first clip arm and the second clip arm, such that the first and second clip arms engage the port collar and the spike collar and such that the claw arms engage a main body of the infusion adapter.

In certain configurations, the securing mechanism may include a catch clip including a flat extension member having a first end and a second end, a first C-shaped clip affixed to the first end of the flat extension member, and a second C-shaped clip affixed to the second end of the flat extension member. The first and second C-shaped clips may be spaced apart by the flat extension member such that, when the catch clip is engaged with the port and the main spike portion, the first C-shaped clip is positioned above the port collar and the second C-shaped clip is positioned below the spike collar, and the first and second C-shaped clips may include flexible clips configured to engage the port and the main spike portion via an interference fit.

In certain configurations, the infusion adapter includes a pair of stems positioned at the first end of the infusion adapter and on opposing sides of the connection spike, and the securing mechanism may include a bayonet clip including a pair of C-shaped clip halves joined together via a flexible hinge, so as to be movable from an open position to a closed position, each of the pair of C-shaped clip halves including an L-shaped slot formed therein configured to receive a respective one of the pair of stems therein. With the bayonet clip positioned about the engaged port and spike portion, the bayonet clip may be locked in place relative to the infusion adapter via engagement of the stems with the L-shaped slots.

In certain configurations, the bayonet clip further includes a locking mechanism via which the C-shaped clip halves may be locked together to secure the bayonet clip in the closed position about the engaged port and spike portion. The securing mechanism may include a spring clamp including an annular holder defining an opening sized to receive the engaged port and spike portion therein, and first and second actuation arms extending out from the annular holder in a crossed arrangement, with the first actuation arm and the second actuation arm being vertically offset to allow for crossing of the first and second actuation arms. Each of the first and second actuation arms may include a thumb press via which the first and second actuation arms may be actuated. The first and second locking arms may be joined to the first and second actuation arms, respectively, with the first and second locking arm extending toward each other, such that the first and second locking arms may selectively engage each other based on positioning of the first and second actuation arms, wherein each of the first and second locking arms includes a plurality or teeth formed thereon, with the first locking arm having teeth on a top surface thereof and the second locking arm having teeth on a bottom surface thereof, such that the teeth may engage each other.

In certain configurations, the annular holder is retained in an initial expanded position via engagement of the teeth on the first and second locking arms, and wherein upon application of a pinching force to the thumb presses, the teeth of the first and second locking arms are brought out of engagement, allowing the first and second actuation arms to separate further, and moving the annular holder from the expanded position to a tightened position, where the spring clamp applies a compression force against the engaged port and main spike portion to prevent dislodging of the main spike portion from the port.

### BRIEF DESCRIPTION OF THE DRAWINGS

FIG. 1 is a perspective view of an infusion adapter with which aspects of the disclosure may be implemented;

FIG. 2 is a right side view of the infusion adapter of FIG. 1;

FIG. 3 is a bottom view of the infusion adapter of FIG. 1;

FIG. 4 s a cross-sectional view along line 4-4 shown in FIG. 3;

FIG. 5 is a front view of the infusion adapter of FIG. 1;

FIG. 6 is a rear view of the infusion adapter of FIG. 1;

FIG. 7 is a cross-sectional view along line 4-4 shown in FIG. 3, showing the infusion adapter inserted into a port of an infusion fluid container;

FIG. 8 is a perspective view of another infusion adapter with which aspects of the disclosure may be implemented;

FIG. 9 is a right side view of another infusion adapter with which aspects of the disclosure may be implemented;

FIG. 10 is a top view of a clamp for securing a port of an infusion fluid container together with a spike portion of an infusion adapter, according to an aspect of the disclosure, with the clamp shown in an open configuration;

FIG. 11 is a top view of the clamp of FIG. 10, with the clamp shown in a closed configuration;

FIG. 12 is a perspective view of the clamp of FIG. 10 shown secured to an engaged infusion fluid container port and infusion adapter spike portion;

FIG. 13 is a top view of a clamp for securing a port of an infusion fluid container together with a spike portion of an infusion adapter, according to another aspect of the disclosure, with the clamp shown in an open configuration;

FIG. 14 is a top view of the clamp of FIG. 13, with the clamp shown in a closed configuration;

FIG. 15 is a perspective view of the clamp of FIG. 13 shown secured to an engaged infusion fluid container port and infusion adapter spike portion;

FIG. 16 is a top view of a clamp for securing a port of an infusion fluid container together with a spike portion of an infusion adapter, according to another aspect of the disclosure, with the clamp shown in an open configuration;

FIG. 17 is a top view of the clamp of FIG. 16, with the clamp shown in a closed configuration;

FIG. 18 is a perspective view of the clamp of FIG. 16 shown secured to an engaged infusion fluid container port and infusion adapter spike portion;

FIG. 19 is a perspective view of a clamp for securing a port of an infusion fluid container together with a spike portion of an infusion adapter, according to another aspect of the disclosure;

FIG. 20 is a perspective view of the clamp of FIG. 19 shown secured to an engaged infusion fluid container port and infusion adapter spike portion;

FIG. 21 is a perspective view of a clamp for securing a port of an infusion fluid container together with a spike portion of an infusion adapter, according to another aspect of the disclosure;

FIG. 22 is a perspective view of a clamp for securing a port of an infusion fluid container together with a spike portion of an infusion adapter, according to another aspect of the disclosure;

FIG. 23 is a perspective view of the clamp of FIG. 21 and/or FIG. 22 shown secured to an engaged infusion fluid container port and infusion adapter spike portion;

FIG. 24 is a cross-sectional view of FIG. 23;

FIG. 25 is a perspective view of a clamp for securing a port of an infusion fluid container together with a spike portion of an infusion adapter, according to another aspect of the disclosure, with the clamp shown in a closed configuration;

FIG. 26 is a perspective view of the clamp of FIG. 25, with the clamp shown in an open configuration;

FIG. 27 is a perspective view of a clamp for securing a port of an infusion fluid container together with a spike portion of an infusion adapter, according to another aspect of the disclosure, with the clamp shown in a closed configuration;

FIG. 28 is a cross-sectional view of the clamp of FIG. 27 shown secured to an engaged infusion fluid container port and infusion adapter spike portion;

FIG. 29 is a perspective view of a clamp for securing a port of an infusion fluid container together with a spike portion of an infusion adapter, according to another aspect of the disclosure, with the clamp shown in an unassembled configuration;

FIG. 30 is a perspective view of the clamp of FIG. 29 positioned relative to an engaged infusion fluid container port and infusion adapter spike portion;

FIG. 31 is a perspective view of the clamp of FIG. 29 shown secured to an engaged infusion fluid container port and infusion adapter spike portion;

FIG. 32 is a cross-sectional view of FIG. 31;

FIG. 33 is a perspective view of a clip for securing a port of an infusion fluid container together with a spike portion of an infusion adapter, according to another aspect of the disclosure;

FIG. 34 is a perspective view of the clamp of FIG. 33 shown secured to an engaged infusion fluid container port and infusion adapter spike portion;

FIG. 35 is a top view of a clip for securing a port of an infusion fluid container together with a spike portion of an infusion adapter, according to another aspect of the disclosure;

FIG. 36 illustrates actuation of the clip of FIG. 35 from an open configuration to a closed configuration;

FIG. 37 is a perspective view of a clip for securing a port of an infusion fluid container together with a spike portion of an infusion adapter, according to another aspect of the disclosure;

FIG. 38 is a perspective view of a clip for securing a port of an infusion fluid container together with a spike portion of an infusion adapter, according to another aspect of the disclosure, with the clamp shown in an open configuration;

FIG. 39 is a perspective view of the clip of FIG. 38, with the clamp shown in a closed configuration;

FIG. 40 is a perspective view of the clip of FIGS. 38 and 39 shown secured to an engaged infusion fluid container port and infusion adapter spike portion;

FIG. 41 is a perspective view of a clip for securing a port of an infusion fluid container together with a spike portion of an infusion adapter, according to another aspect of the disclosure;

FIG. 42 is a perspective view of the clamp of FIG. 41 shown secured to an engaged infusion fluid container port and infusion adapter spike portion;

FIG. 43 is a perspective view of a clip for securing a port of an infusion fluid container together with a spike portion of an infusion adapter, according to another aspect of the disclosure;

FIG. 44 is another perspective view of the clip of FIG. 43;

FIG. 45 is a perspective view of the clamp of FIGS. 43 and 44 shown secured to an engaged infusion fluid container port and infusion adapter spike portion;

FIG. 46 is a perspective view of a clamp for securing a port of an infusion fluid container together with a spike portion of an infusion adapter, according to another aspect of the disclosure;

FIG. 47 is a top view of the clamp of FIG. 46, with the clamp shown in an expanded position;

FIG. 48 is a top view of the clamp of FIG. 46, with the clamp shown in a tightened position; and

FIG. 49 is a perspective view of the clamp of FIG. 46 shown secured to an engaged infusion fluid container port and infusion adapter spike portion.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

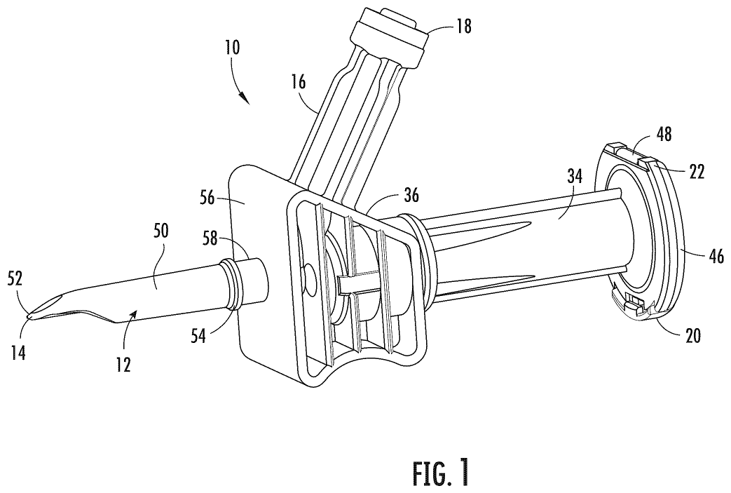

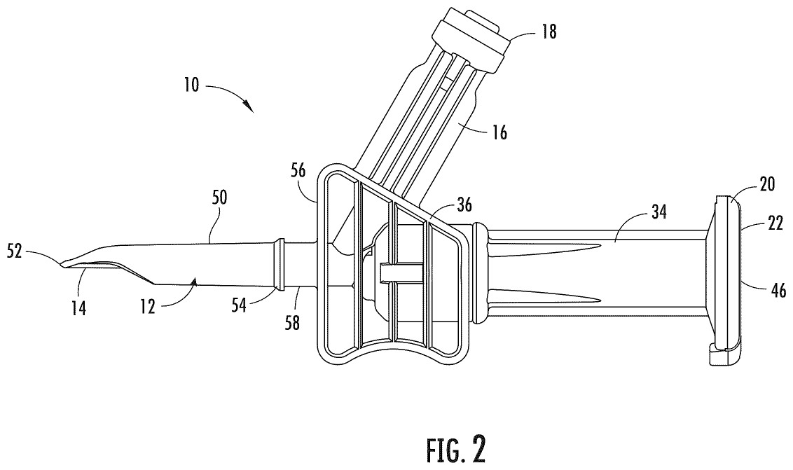

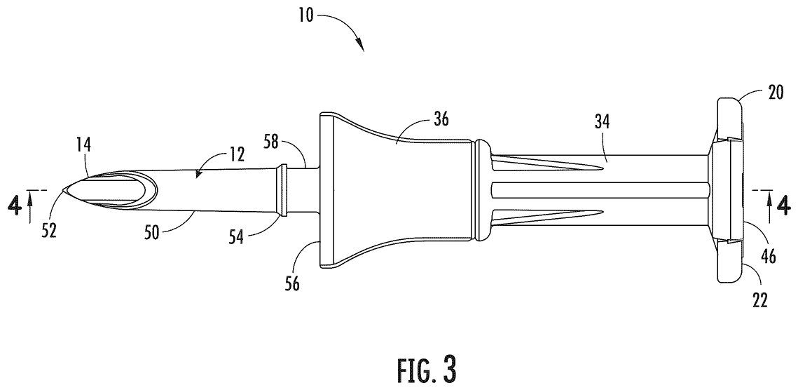

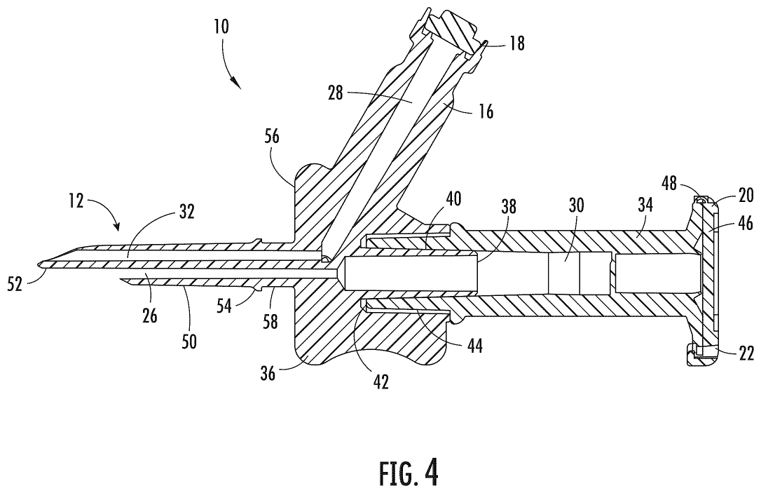

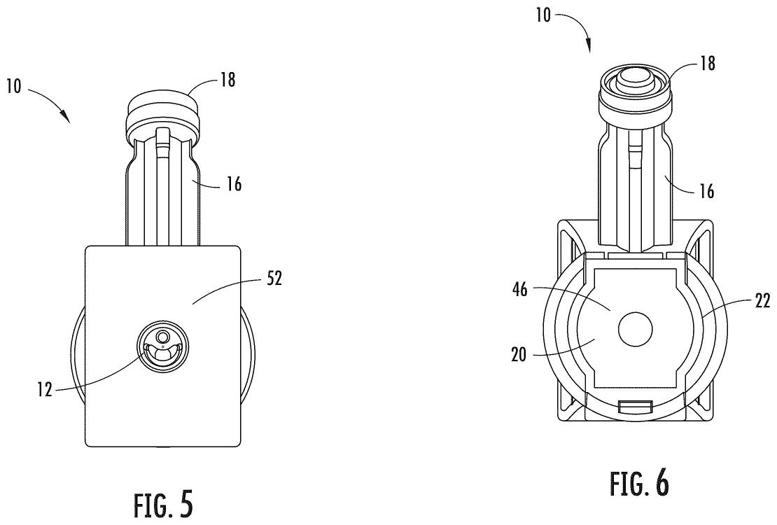

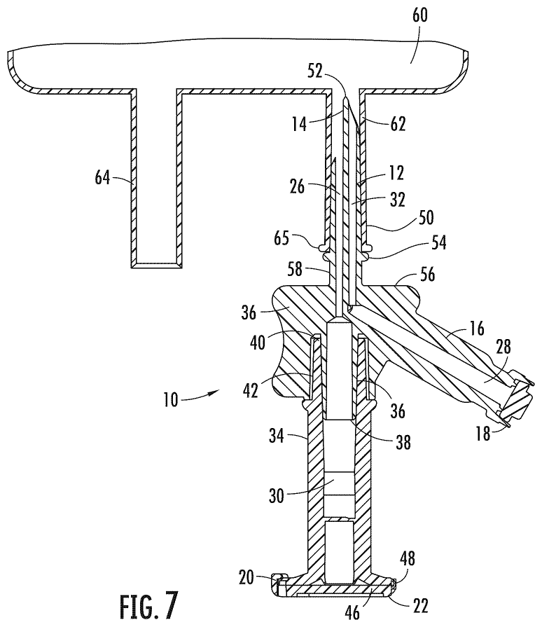

Referring to FIGS. 1-7, shown is a non-limiting embodiment of an infusion adapter 10 used in/with the closed preparation of a drug. In one embodiment, infusion adapter 10 may be structured similarly to a PhaSeal™M adapter which is compatible with a Becton Dickinson (“BD”) PhaSeal™ System available from Becton, Dickinson and Company of Franklin Lakes, N.J. The infusion adapter 10 thus includes a connection portion 12 located at a first end 14, a first port 16 located at a first port end 18, and a second port 20 located at a second port end 22. Connection portion 12 includes a fluid channel 26 and a fluid channel 32, although only a single channel arrangement may also be utilized. First port 16 includes a first port fluid channel 28 and second port 20 includes a second port fluid channel 30. As shown more clearly in FIGS. 4 and 7, the fluid channel 32 of connection portion 12 is in fluid communication with first port fluid channel 28 of first port 16 such that a fluid may flow into infusion adapter 10 at first port 16, travel through first port fluid channel 28 to fluid channel 32 of connection portion 12 and out first end 14 of infusion adapter 10. The fluid channel 26 of connection portion 12 is in fluid communication with second port fluid channel 30 of second port 20 such that a fluid may flow into infusion adapter 10 at first end 14 of connection portion 12, travel through fluid channel 26 to second port fluid channel 30 and out second port 20 of infusion adapter 10.

As shown in FIGS. 1-7, in one embodiment, infusion adapter 10 may comprise a generally Y-shape. Further, it is contemplated that infusion adapter 10 may be made available in a variety of shapes and sizes as long as first port 16 is spaced a distance from second port 20 so that first port 16 may be connected to a syringe assembly containing a medication fluid and second port 20 may be connected to an intravenous line that is adapted for connection to a bloodstream of a patient as will be described in more detail below. For example, in another embodiment, infusion adapter 10 may comprise a generally T-shape.

Referring to FIG. 4, in one embodiment, infusion adapter 10 may include an intravenous line connector 34 that is removably connectable to a main body 36 of infusion adapter 10. In such an embodiment, main body 36 includes an intravenous line connector receiving end 38 having a first connection portion 40. Additionally, intravenous line connector 34 includes a main body receiving end 42 having a second connection portion 44. Intravenous line connector 34 includes an end cap 46 that is pivotable via a hinge portion 48 between an open position and a closed position.

Intravenous line connector 34 may be connected to main body 36 by positioning main body receiving end 42 of intravenous line connector 34 into engagement with intravenous line connector receiving end 38 of main body 36. In one embodiment, intravenous line connector 34 may be secured to main body 36 by positioning second connection portion 44 of intravenous line connector 34 into engagement with first connection portion 40 of main body 36, and threadingly engaging first connection portion 40 and second connection portion 44. In other embodiments, second connection portion 44 of intravenous line connector 34 may be secured to first connection portion 40 of main body 36 using a press-fit, locking tapers, interference fit, snap-fit, ball detent, locking tabs, spring loaded locking mechanism, latch, adhesive, or other similar mechanism. In this manner, intravenous line connector 34 is locked to main body 36, i.e., significant relative movement between intravenous line connector 34 and main body 36 is prevented. In alternate embodiments, intravenous line connector 34 and main body 36 may be integrally formed.

According to aspects of the disclosure, connection portion 12 of infusion adapter 10 includes an elongated cylindrical main spike portion 50, a puncturing point 52 disposed adjacent first end 14, and an annular spike collar 54 provided on main spike portion opposite from puncturing point 52.

The fluid channels 26, 32 extend through a length of the main spike portion 50—extending from the puncturing point 52 at first end 14 to a junction/connection with first port fluid channel 28 and/or second port fluid channel 30.

The puncturing point 52 comprises a sharpened point or end that is configured to engage and pierce into an infusion fluid container. In one example, the puncturing point 52 is configured to be inserted/spiked to an IV bag through a rubber bung in an IV bag port.

The spike collar 54 extends radially outward from the main spike portion 50 and is positioned adjacent to a stop 56 defined by the main body 36 of the infusion adapter 10. A space 58 is provided between the spike collar 54 and the stop 56. In some embodiments, a plurality of reinforcement ribs (not shown) may be formed about main spike portion 50 in the space 58 between the spike collar 54 and the stop 56 that may be spaced circumferentially about main spike portion 50 and extend radially outward therefrom to provide a connection of increased strength between the connection portion 12 and the main body 36.

As previously discussed, intravenous therapy applications allow patients to receive infusion and medication treatment. For example, therapy may include the administration of medications by IV using intravenous and subcutaneous or hypodermis routes, i.e., into the bloodstream and under the skin. Examples of medical treatments that intravenous therapy applications may provide to a patient include antibiotics, pain management medications, cancer treatments, and similar medications.

Referring to FIG. 7, infusion adapter 10 is shown connected to an infusion fluid container 60, which may comprise an IV bag according to one embodiment. More specifically, the connection portion 12 of the infusion adapter 10 is inserted into a first or injection port 62 of the infusion fluid container 60. In some embodiments, the infusion fluid container 60 may also include a second port 64. In some embodiments, the injection port 62 of infusion fluid container 60 may include an annular port collar 65 thereon, with the annular port collar 65 positioned toward an end of the port 62, such that it may be brought adjacent to spike collar 54 upon engagement of the main spike portion 50 with the port 62.

With infusion adapter 10 securely connected to port 62 of infusion fluid container 60, a patient or a medical practitioner is able to connect a syringe assembly (not shown) to first port 16 of infusion adapter 10. The first port 16 may be compatible with any suitable connection arrangement and any suitable syringe adapter. With the syringe assembly connected to first port 16, a medication fluid contained in the syringe assembly can be injected into the infusion fluid container 60 via infusion adapter 10. The syringe assembly may then be disconnected from infusion adapter 10 and the infusion fluid container 60 may then be sent to nursing and is ready to be administered to a patient. For example, an intravenous line or intravenous tubing may be connected to second port 20 of infusion adapter 10 with the other end of the intravenous tubing connected to a bloodstream of a patient. In this manner, a medication may be administered to the patient intravenously.

While the infusion adapter 10 shown in FIGS. 1-7 is configured as a Y-shaped adapter that includes an intravenous line connector 34 for connection with an intravenous line/tube, it is recognized that infusion adapters could be provided having different configurations, according to further aspects of the disclosure.

Referring to FIG. 8, an infusion adapter 10A, according to a second aspect, includes a direct connection with intravenous tubing 66 rather than providing the intravenous line connector 34 shown in FIGS. 1-7.

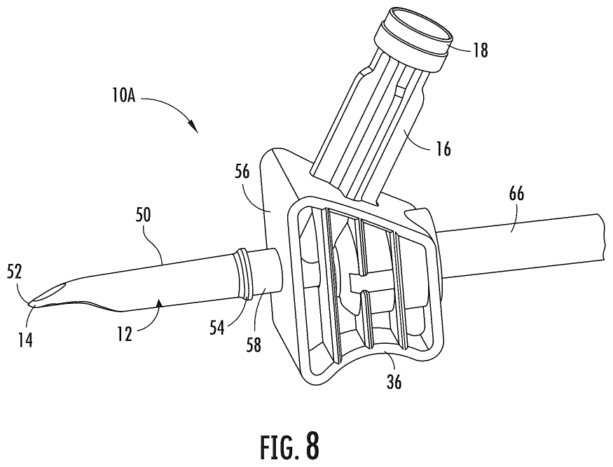

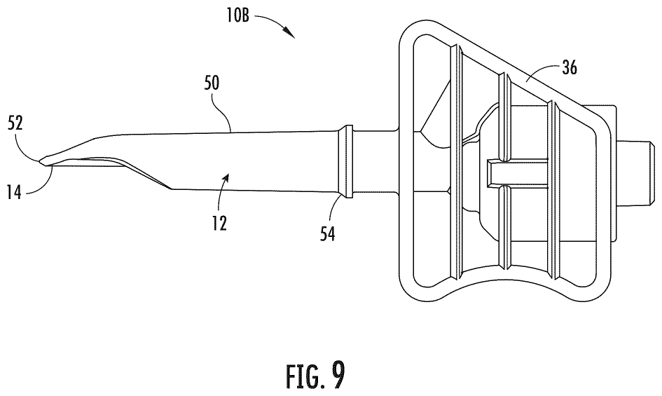

Referring to FIG. 9, an infusion adapter 10B, according to a third aspect, includes a direct connection for intravenous tubing and does not include the first port 16 shown in FIGS. 1-7.

In any of the infusion adapters 10, 10A, 10B shown in FIGS. 1-9, it is recognized that it is desirable for the connection portion 12 to be securely retained within the port 62 of the infusion fluid container 60, in order to prevent inadvertent and accidental dislodging of the connection portion 12 out from the port 62 during a drug transfer procedure.

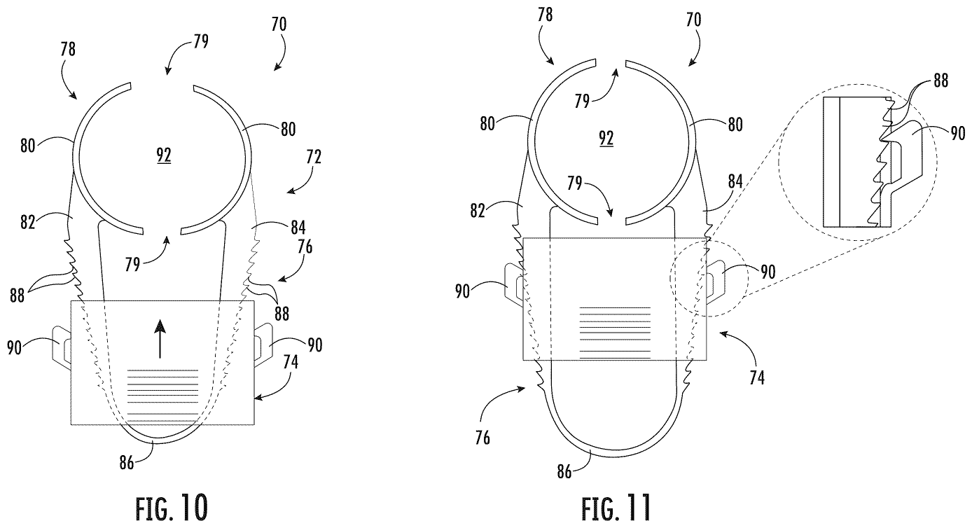

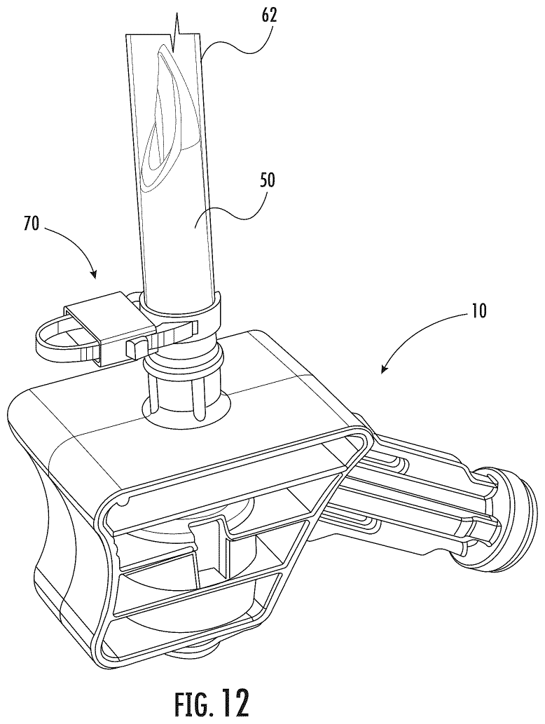

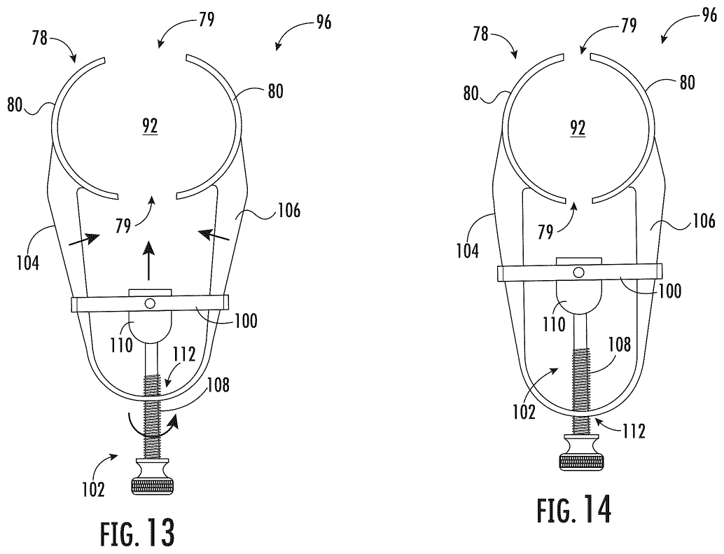

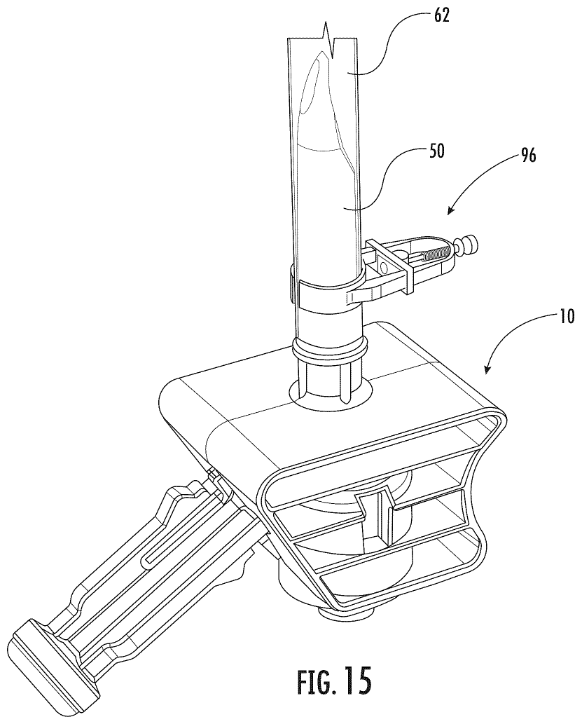

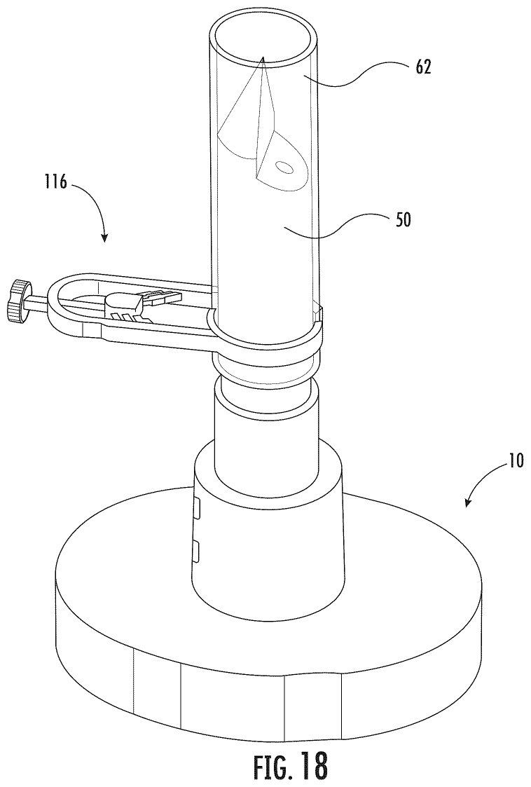

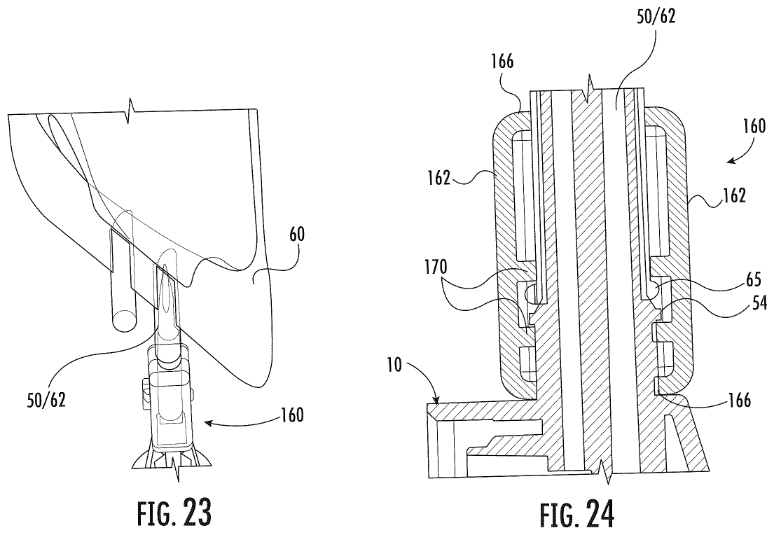

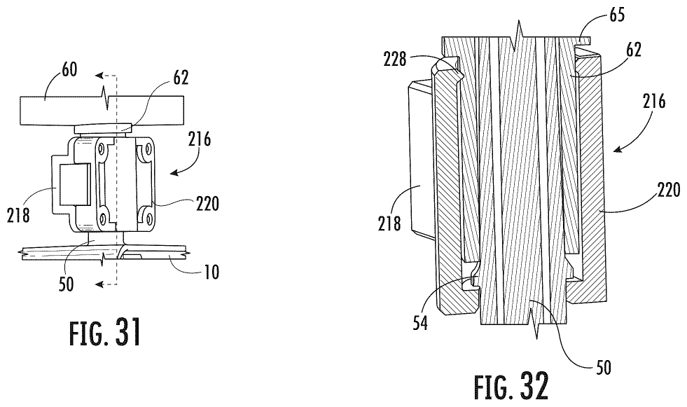

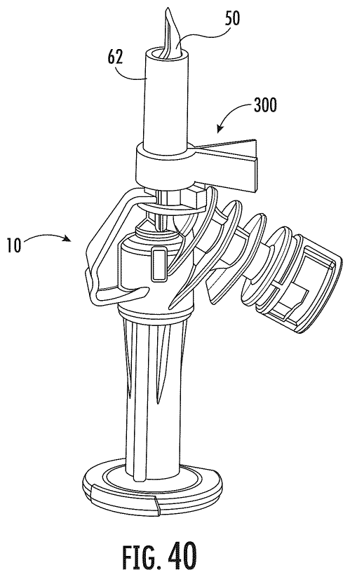

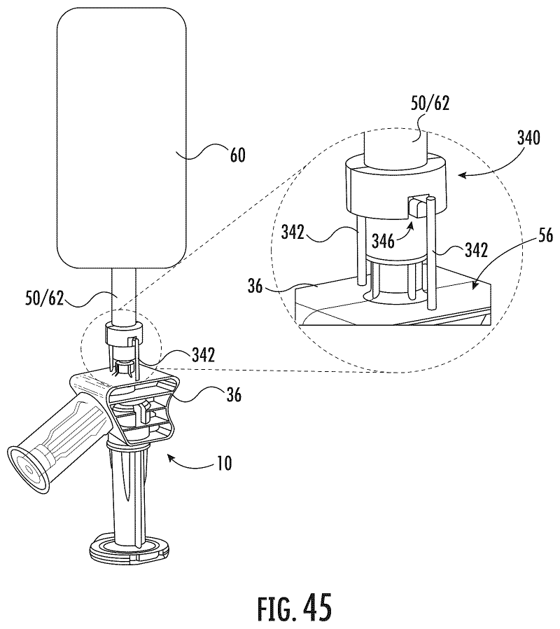

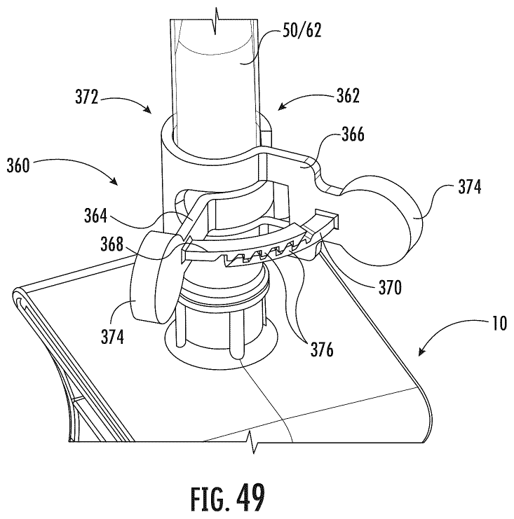

According to aspects of the disclosure, a separate or add-on securing mechanism may be provided for use with the adapter 10 and infusion fluid container 60 to maintain engagement of the connection portion 12 with the port 62, so as to prevent leaking of medication from the infusion fluid container 60 that could lead to hazardous drug exposure and/or costly medication waste. Embodiments of various add-on securing mechanisms are provided in FIGS. 10-49 and are described in more detail here below.

Referring to FIGS. 10-32, various clamp-type securing mechanisms that may be attached/added after mating of the adapter 10 and infusion fluid container 60 are illustrated, according to embodiments of the disclosure. In some embodiments, the clamp-type securing mechanism may be applied onto the port 62 and main spike portion 50 after engagement thereof, with the clamp-type securing mechanism subsequently tightened to secure the port 62 and spike portion 50 together.

In the embodiment of FIGS. 10-12, a clamp 70 is illustrated that is configured as a hinge clamp. The hinge clamp 70 is generally composed of a clamp member 72 and a slider 74 that operates with the clamp member 72 to tighten the clamp member 72. The clamp member 72 is formed as a singular member that includes a flexible leg assembly 76 and an annular-shaped holder 78 with slits 79 formed therein to separate the holder 78 into a pair of holder portions 80 that may be selectively joined and separated. The holder 78 is configured to be positioned about the port 62 and spiked portion 50 after pushing of the spike portion 50 into port 62 and may be tightened so as to secure the adapter 10 to IV bag.

Flexible leg assembly 76 is formed as a U-shaped member having a first leg 82 and a second leg 84. The first leg 82 and second leg 84 are joined together at a first end thereof by a connecting portion 86, while a respective holder portion 80 is joined to each of the first leg 82 and second leg 84 at a second end thereof. Each of the first leg 82 and second leg 84 is biased outward, with the connecting portion 86 being flexible and functioning as a hinge about which the first leg 82 and second leg 84 may pivot-with the legs being pivotable inwardly when an inward-directed force is applied thereto. Each of the first leg 82 and second leg 84 includes a plurality or serrations or teeth 88 formed on an outer facing surface thereof, with the teeth 88 extending generally from the first end to the second end of the leg 82, 84. The teeth are 88 configured as angled teeth that are angled toward the second end of the legs 82, 84, so as to allow movement of the slider 74 relative to clamp member 72 in only one direction, as explained in further detail below.

The slider 74 of clamp 70 is configured as a hollow, box-shaped member that may be positioned about the first leg 82 and second leg 84 of the clamp member 72. The slider 74 is translatable relative to the clamp member 72, with a pair of flexible tongues 90 provided on the slider 74 that are configured to interact with the teeth 88 on the first and second legs 82, 84 to control movement of the slider 74. The interaction of the flexible tongues 90 with teeth 88 is such that movement of the slider 74 from the first end of legs 82, 84 toward the second end is allowed, while movement of the slider 74 from the second end of legs 82, 84 toward the first end is prevented. The slider 74 applies an inward-directed force against the legs 82, 84 when the slider 74 is moved from the first end of legs toward the second end of legs.

In operation of the clamp 70, as the slider 74 is moved along the clamp member 72 from the first end of legs 82, 84 toward the second end, the slider 74 acts to pinch the first leg 82 and second leg 84 together, such that a gap between the first leg 82 and the second leg 84 is reduced. As the first leg 82 and second leg 84 are pinched together, the holder portions 80 of the clamp member 72 are also brought closer together, with a size of the slits 79 formed in holder 78 being reduced. As the holder portions 80 are brought closer together, a size of an opening or receptacle 92 defined thereby is reduced.

In using the clamp 70 to secure the adapter 10 to the infusion fluid container 60, the spike portion 50 is initially pushed into port 62 to engage the adapter 10 with the infusion fluid container 60. Once the spike portion 50 is completely inserted into the port 62, the clamp 70 is provided, with the holder 78 positioned about the port 62 and spike member 50 (i.e., port 62 and spike member 50 may be pushed through a slit 79 formed in holder 78, such that holder 78 is positioned thereabout). Once holder 78 is positioned about the port 62 and spike member 50, the slider 74 may be actuated by a user and pushed from the first end of legs 82, 84 toward the second end of legs. As the slider 74 is moved, the slider 74 acts to pinch the first leg 82 and second leg 84 together, with the holder portions 80 of the clamp member 72 thus also being brought closer together such that the size of the opening/receptacle 92 defined by the holder 78 is reduced. As the size of opening/receptacle 92 is reduced, the holder portions 80 apply a compression force against the port 62 and the spike portion 50 positioned therein that prevents dislodgement of the spike portion 50 from the port 62, such that adapter 10 remains connected to infusion fluid container 60. The holder 78 is retained or locked in this tightened condition/position due to the interaction of flexible tongues 90 and teeth 88, as the slider 74 is prevented from moving from the second end of legs 82, 84 toward the first end of legs.

In the embodiment of FIGS. 13-15, a hinge clamp 96 is illustrated that is generally composed of a clamp member 98, a slider 100 that operates with the clamp member 98 to tighten the clamp member, and a screw adjuster 102 that is used to actuate the slider 100. The clamp member 98 is similar to clamp member 98 shown/described for FIGS. 10-12, and is thus not described in detail again, except that a first leg 104 and second leg 106 of clamp member 98 do not include teeth formed thereon—with outward facing surfaces of the first leg 104 and second leg 106 instead being smooth, so as to allow for a smooth movement of the slider 100 relative to clamp member 98.

The slider 100 of hinge clamp is structured as a rectangular member that defines a hollow interior, with the slider 100 positionable about the first leg 104 and second leg 106 of the clamp member 98. The slider 100 is translatable relative to the clamp member 98, with the slider 100 applying an inward-directed force against the legs 104, 106 when the slider 100 is moved from the first end of legs toward the second end of legs.

The screw adjuster 102 comprises a thumb screw 108 and a slider connector 110. The thumb screw 108 is inserted through a hole 112 in connecting portion of clamp member 98 and may be linearly advanced through the hole 112 (and relative to clamp member 98) via rotation thereof. The slider connector 110 is provided at an end of the thumb screw 108 and as joined to the slider 100, such that linear movement of the thumb screw 108 relative to clamp member 98 is transferred to slider 100-with the slider 100 thus moving linearly relative to clamp member 98 responsive to rotation of the thumb screw 108. The slider connector 110 is joined to thumb screw 108 such that the thumb screw 108 rotates freely relative to the slider connector 110, with only linear movement of the thumb screw 108 being transferred to the slider connector 110.

In using the clamp 96 to secure the adapter to the infusion fluid container 60, the spike portion 50 is initially pushed into port 62 to engage the adapter 10 with the infusion fluid container 60. Once the spike portion 50 is completely inserted into the port 62, the clamp 96 is provided, with its holder 78 positioned about the port 62 and spike member 50 (i.e., port 62 and spike member 50 may be pushed through a slit 79 formed in holder 78, such that holder 78 is positioned thereabout). Once holder i78 s positioned about the port 62 and spike member 50, the thumb screw 108 may be actuated by a user—with rotation of the thumb screw 108 causing a liner movement of the thumb screw 108 relative to clamp member 98. Linear movement of the thumb screw 108 is transferred to slider 100, with the slider 100 moving from the first end of legs 104, 106 toward the second end of legs. As the slider 100 is moved, the slider 100 acts to pinch the first leg 104 and second leg 106 together, with the holder portions 80 of the clamp member 98 thus also being brought closer together such that the size of the opening/receptacle 92 defined by the holder 78 is reduced. As the size of opening/receptacle 92 is reduced, the holder portions 80 apply a compression force against the port 62 and the spike portion 50 positioned therein that prevents dislodgement of the spike portion 50 from the port 62, such that adapter 10 remains connected to infusion fluid container 60. The holder 78 is retained or locked in this tightened condition/position due to the setting of the thumb screw 108 in its position relative to clamp member 98.

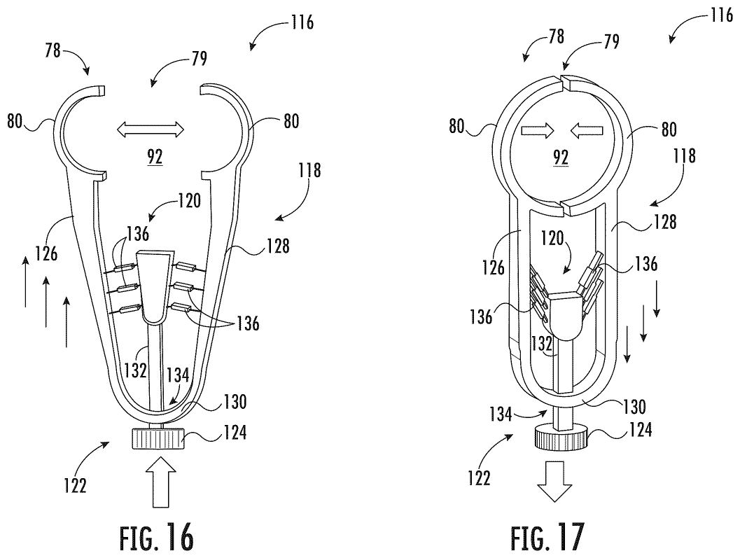

In the embodiment of FIGS. 16-18, a hinge clamp is illustrated that is configured as a bi-stable hinge clamp 116. The bi-stable hinge clamp 116 is generally composed of a clamp member 118, a live hinge 120, and an actuator 122 including a button 124 thereon for operating the live hinge 120.

The clamp member 118 includes a first leg 126 and second leg 128 joined via a connecting portion 130. The clamp member 118 also includes a holder 78 including holder portions 80, similar to as previously described for the clamps 70, 96 of FIGS. 10-12 and FIGS. 13-15.

The actuator 122 comprises a shaft 132 that is inserted through a hole 134 in connecting portion 130 of clamp member 118, with the button 124 provided at a first end of shaft 132. The live hinge 120 is provided at a second end of shaft 132, with the live hinge 120 comprising a plurality of hinge members 136 that extend between the shaft 132 and one of the first leg 126 and second leg 128 of clamp member 118. The hinge members 136 of live hinge 120 are flexible members that are actuatable from an extended configuration to a collapsed configuration responsive to operation of actuator 122 by a user. That is, with the actuator 122 in a first position, the hinge members 136 of live hinge 120 are in an extended configuration that positions the first leg 126 and second leg 128 of clamp member 118 in an “open” stable equilibrium position, with holder portions 80 thus separated from one another by a large distance. With the actuator 122 in a second position, with button 124 being pulled away from holder 78, the hinge members 136 of live hinge 120 are in a collapsed configuration that positions the first leg 126 and second leg 128 of clamp member 118 in a “closed” stable equilibrium position, with holder portions 80 thus moved together and into contact with each other (or separated by a very small slit/gap 79). In some embodiments, a bump or snap (not shown) may be provided on clamp member 118 that prevents accidental movement of the actuator 122 from the second position back to the first position, which would cause the hinge clamp 116 to open.

In using the clamp 116 to secure the adapter to the infusion fluid container 60, the spike portion 50 is initially pushed into port 62 to engage the adapter 10 with the infusion fluid container 60. Once the spike portion 50 is completely inserted into the port 62, the clamp 116 is provided, with the holder 78 positioned about the port 62 and spike member 50 (i.e., port 62 and spike member 50 may be pushed through a slit 79 formed in holder 78, such that holder 78 is positioned thereabout). Once holder 78 is positioned about the port 62 and spike member 50, the button 124 may be actuated (i.e., pulled) by a user from a first position to a second position—with movement of the button 124 (and actuator 122) causing actuation of the live hinge 120, i.e., actuation of hinge members 136 from the extended configuration to the collapsed configuration. Actuation of the live hinge 120 causes the clamp member 118 to move from an “open” stable equilibrium position to a “closed” stable equilibrium position, with the holder portions 80 of the clamp member 118 being brought together such that the size of the opening/receptacle 92 defined by the holder 78 is reduced. As the size of opening/receptacle 92 is reduced, the holder portions 80 apply a compression force against the port 62 and the spike portion 50 positioned therein that prevents dislodgement of the spike portion 50 from the port 62, such that adapter 10 remains connected to infusion fluid container 60. The holder 78 may be retained or locked in this tightened condition/position via a bump or snap on clamp member 118 locking the actuator/button 122, 124 in position.

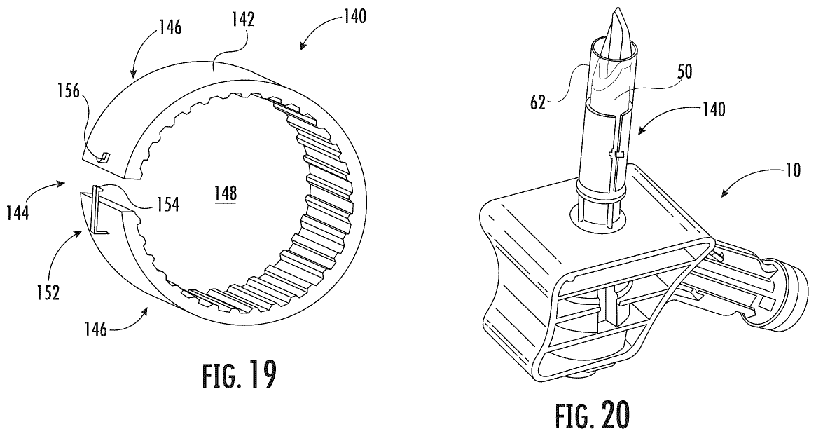

In the embodiment of FIGS. 19 and 20, a clamp is illustrated that is configured as a sleeve-type clamp 140. The clamp 140 comprises a cylindrical sleeve 142 with a slit 144 formed therein that separates the sleeve 142 into a pair of flexible sleeve portions 146 that may deflect when a force is applied thereto. The sleeve 142 is configured to be positioned about the port 62 and spiked portion 50 after pushing of the spike portion 50 into port 62, so as to secure the adapter 10 to infusion fluid container 60. That is, when the sleeve 142 is pushed into contact with the port 62 and spike portion 50 at the location of slit 144, the slit 144 is caused to expand and the flexible sleeve portions 146 separate, such that the port 62 and spike portion 50 may be pushed into an opening or receptacle 148 defined by the sleeve clamp 140. The clamp 140 is configured to be positioned about the port 62 and spiked portion 50 after pushing of the spike portion 50 into port 62 and may be tightened so as to secure the adapter 10 to infusion fluid container 60.

In some embodiments, an inner surface of the sleeve 142 may include a plurality of teeth 150 spaced circumferentially about the inner surface, with each of the teeth 150 extending radially inward. The teeth 150 provide additional grip/friction between the clamp 140 and the port 62/spike portion 50, to prevent movement of the clamp 140 relative thereto.

The sleeve clamp 140 may include a locking mechanism 152 via which the portions of the sleeve 142 may be locked together to secure the sleeve clamp 140 in place about the port 62 and spike portion 50. In an exemplary embodiment, the sleeve portions 146 of the clamp 140 may be locked together via a tongue and groove type lock mechanism. That is, one sleeve portion 146 of the clamp 140 may include a tab or tongue 154 formed at the end thereof, while the other sleeve portion 146 of the clamp 140 may include a hook or groove 156 formed therein configured to receive the tongue 154 therein. A user may push the tongue 154 into engagement with the hook/groove 156 upon positioning of the sleeve clamp 140 about the port 62 and spike portion 50, to thus lock the sleeve clamp 140 in a closed configuration. With the sleeve clamp 140 in the closed configuration, the portions of the sleeve clamp 140 apply a compression force against the port 62 and the spike portion 50 that prevents dislodgement of the spike portion 50 from the port 62, such that adapter 10 remains connected to infusion fluid container 60.

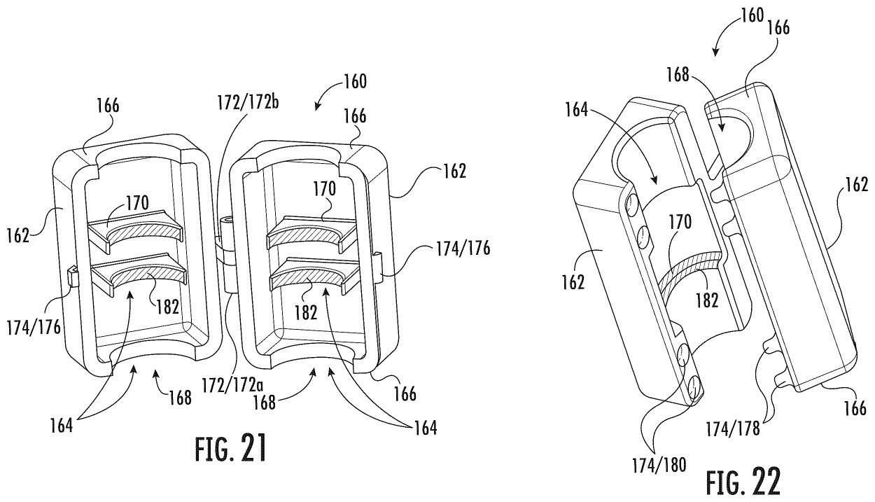

Referring to FIGS. 21-32, various enclosure-type securing mechanisms that may be attached/added after mating of the infusion adapter 10 and infusion fluid container 60 are illustrated, according to embodiments of the disclosure. In some embodiments, the enclosure-type securing mechanism may be applied onto the port 62 and spike portion 50 after engagement thereof, with the enclosure-type securing mechanism engaging with the port 62 and spike portion 50 to secure the adapter 10 and infusion fluid container 60 together.

In the embodiments of FIGS. 21-24, a hinge lock enclosure clamp 160 is illustrated. The hinge lock enclosure clamp 160 comprises a pair of separate brackets 162 that are hinged together. In some embodiments, each of the brackets 162 may have a box like shape such that, when the brackets 162 joined together, a hollow interior is defined. In other embodiments, each of the brackets 162 may have a barrel shape. Each of the brackets 162 comprises a C-shaped channel 164 defined therein that extends a length of the bracket, with opposing end walls 166 of the bracket 162 having semi-circular cut-out 168 formed therein and C-shaped protrusions 170 being provided on an inner surface of the bracket 162. The brackets 162 are hinged together via hinge features 172 provided on the brackets 162, which may comprise a hinge pin 172a on one bracket 162 and a receptacle 172b on the other bracket 162 that mates with the hinge pin 172a in a rotating manner.

The hinge lock enclosure clamp 160 may include a locking mechanism 174 via which the brackets 162 may be locked together to secure the hinge lock enclosure clamp 160 in place about the port 62 and spike portion 50.

In the embodiment of FIG. 21, the brackets 162 may be locked together via interacting snaps or tabs 176 configured to engage each other upon the brackets 162 being rotated into contact with each other. A user may push the snaps/tabs 176 into engagement upon positioning of the hinge lock enclosure clamp 160 about the port 62 and spike portion 50, to thus lock the hinge lock enclosure clamp 160 in a closed configuration.

In the embodiment of FIG. 22, the brackets 162 may be locked together via interacting pins 178 and receptacles 180 configured to engage each other upon the brackets 162 being rotated into contact with each other. A user may push the pins 178 of one bracket 162 into the receptacles 180 of the other bracket 162 upon positioning of the hinge lock enclosure clamp 160 about the port 62 and spike portion 50, to thus lock the hinge lock enclosure clamp 160 in a closed configuration.

In some embodiments, each bracket 162 of the hinge lock enclosure clamp 160 may comprise one or more silicone pads 182 thereon at an area of engagement between clamp 160 and the port 62 (and spike portion 50) that acts as a seal to prevent leakage. The silicone pads 182 may be provided on the semi-circular cut-outs 168 and/or C-shaped protrusions 170, with the outer surface of the port 62 engaging the silicone pads 182 when the hinge lock enclosure clamp 160 is closed. In some embodiments, silicone pads 182 may be provided on each of a pair of C-shaped protrusions 170 on each bracket 162, with the C-shaped protrusions 170 spaced about so as to be positioned above and below a spike collar 54 and a port collar 65 of the port 62, such that the spike collar 54 and port collar 65 are sealed between the C-shaped protrusions 170 and the silicone pads 182 thereon.

Beneficially, the hinge lock enclosure clamp 160 will restrict vertical movement between the port 62 and the spike portion 50, so as to prevent dislodgement of the spike portion 50 from the port 62 and ensure that adapter 10 remains connected to infusion fluid container 60.

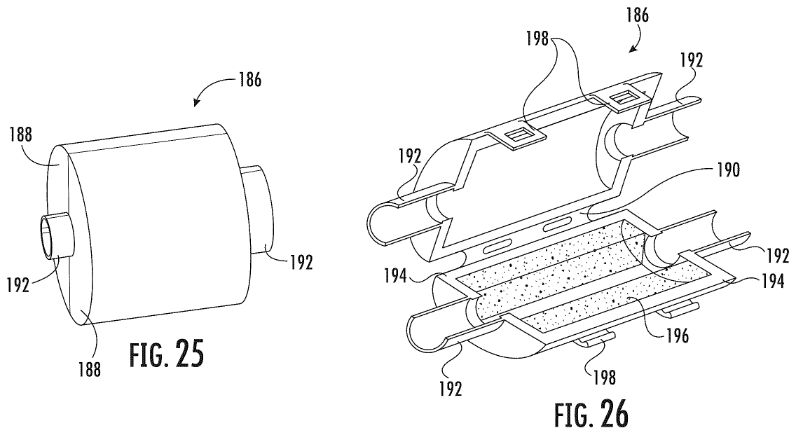

In the embodiment of FIGS. 25 and 26, a hinge lock enclosure clamp 186 is illustrated that is similar to the hinge lock enclosure clamps of FIGS. 21-24. The hinge lock enclosure clamp 186 again is formed of two brackets 188 (e.g., semi-cylindrical brackets) that are joined together via a hinge feature 190. In the hinge lock enclosure clamp 186, a cylindrical sleeve 192 may be provided at each of opposing ends thereof that extends outwardly from a remainder of the bracket 188. Each of the sleeves 192 provides additional contact area between the hinge lock enclosure clamp 186 and port 62/spike portion 50 when the hinge lock enclosure clamp 186 is secured thereto.

In hinge lock enclosure clamp 186, a rubber gasket 194 may be provided on at least one of the brackets 188 and arranged about a perimeter of the bracket 188, where the bracket 188 is brought into engagement with the other bracket 188. The rubber gasket 194 acts as a seal to prevent any leakage from the infusion fluid container 60 (at engagement of the port 62 and spike portion) from escaping the hinge lock enclosure clamp 186.

In some embodiments, an absorbing material 196 may also be included inside the hinge lock enclosure clamp 186 (i.e., within an interior of each bracket 188) that is configured to absorb any leakage of fluid/medicine out from the infusion fluid container 60.

Again, the hinge lock enclosure clamp 186 may include a locking mechanism 198 via which the brackets 188 may be locked together to secure the hinge lock enclosure clamp 186 in place about the port 62 and spike portion 50. In the embodiment of FIGS. 25 and 26, the brackets 188 may be locked together via a locking mechanism 198 comprising an interacting tongue and groove configured to engage each other upon the brackets 188 being rotated into contact with each other, however, an arrangement of snaps or pins and receptacles may instead be included, as previously described.

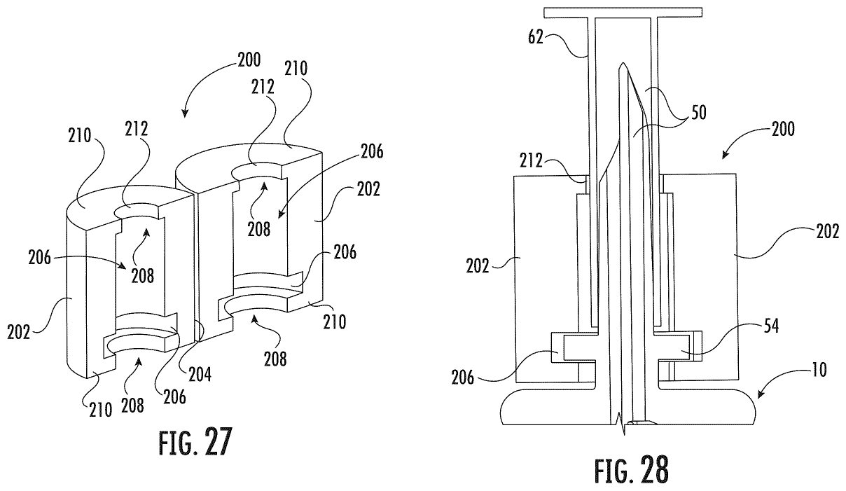

In the embodiment of FIGS. 27 and 28, a hinge lock enclosure clamp 200 is illustrated that is again similar to the hinge lock enclosure clamps 160, 186 of FIGS. 21-24 and FIGS. 25-26. The hinge lock enclosure clamp 200 is formed of two brackets 202 (e.g., semi-cylindrical brackets) that are joined together via a hinge feature 204. Each bracket 202 of the hinge lock enclosure clamp 200 may comprise a C-shaped cut-out or slot 206 formed on an inner surface thereof, such as a location adjacent the bottom end of the bracket 202. The slot 206 is configured to receive the spike collar 54 of spike portion 50 therein. With the spike collar 54 received in slot 206, the hinge lock enclosure clamp 200 is able to restrict vertical movement between the port 62 and the spike portion 50, so as to prevent dislodgement of the spike portion 50 from the port 62 and ensure that adapter 10 remains connected to infusion fluid container 60.

Each bracket of the hinge lock enclosure clamp 200 includes a semi-circular cut-out 208 formed therein at each of opposing end walls 210 of the bracket 202, through which the insertion port 62 and spike portion 50 may pass. As shown in FIG. 27, one or both of these cut-outs 208 (e.g., top cut-out) may have an adhesive 212 applied thereto that aids in securing the hinge lock enclosure clamp 200 to the insertion port 62 and/or spike portion 50—i.e., securing the area where the spike portion 50 is inserted into the infusion fluid container 60. A peel-off cover layer (not shown) may initially be provided over the adhesive 212 and may be removed once the infusion fluid container 60 has been spiked. According to embodiments, any of a number of suitable adhesives may be applied in the semi-circular cut-out(s) 208, with acrylate including methacrylate and epoxy diacrylates (which are also known as vinyl resins) being a non-limiting example.

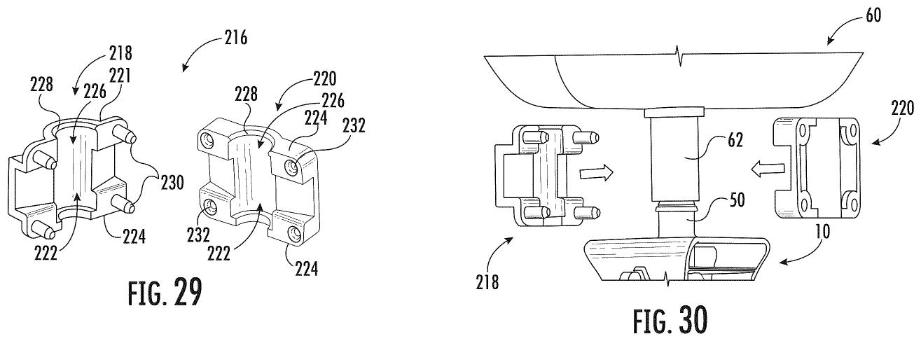

In the embodiment of FIGS. 29-32, an enclosure-type clamp is illustrated that is provided as a press-fit clamp 216. The press-fit clamp 216 comprises a first enclosure tab 218 and a second enclosure tab 220 that, when joined together, define a channel therein that may receive the port 62 and spike portion 50. Each of the enclosure tabs 218, 220 thus comprises a C-shaped channel 222 defined therein that extends a length of the enclosure tab, with opposing end walls 224 of the enclosure tabs 218, 220 having a semi-circular or C-shaped cut-out 226 formed therein. One or more of the semi-circular cut-outs 226 (e.g., the top cut-out) may include a rib 228 formed thereabout the extends radially inward. The rib 228 is configured to engage an outer surface of the port 62 when the press-fit clamp 216 is positioned about the engaged port 62 and spike portion 50 and increase friction between the press-fit clamp 216 and the port 62.

The first enclosure tab 218 and second enclosure tab 220 may be locked together via interacting pins 230 and receptacles 232 configured to engage each other upon the enclosure tabs 218, 220 being moved into contact with each other. A user may push the pins 230 of one enclosure tab into the receptacles 232 of the other enclosure tab upon positioning of the press-fit clamp 216 about the port 62 and spike portion 50, to thus lock the press-fit clamp 216 in a closed configuration.

Beneficially, the press-fit clamp 216 will restrict vertical movement between the port 62 and the spike portion 50, so as to prevent dislodgement of the spike portion 50 from the port 62 and ensure that adapter 10 remains connected to infusion fluid container 60.

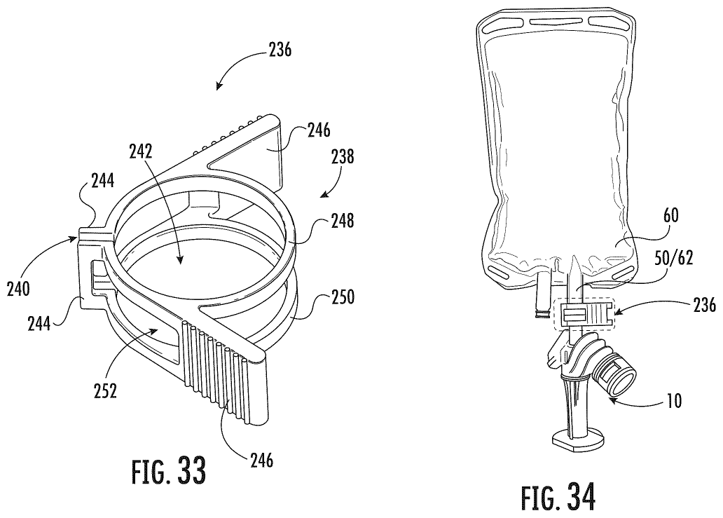

Referring to FIGS. 33-45, various catch clip securing mechanisms that may be attached/added after mating of the infusion adapter 10 and infusion fluid container 60 are illustrated, according to embodiments of the disclosure. In some embodiments, the catch clip securing mechanism may be applied onto the port 62 and spike portion 50 after engagement thereof, with the catch clip securing mechanism subsequently tightened to secure the port 62 and spike portion 50 together.

In the embodiment of FIGS. 33 and 34, a catch clip 236 is illustrated that is formed as a singular member having a generally ring-shaped construction. The catch clip 236 includes an annular-shaped holder 238 having a slit 240 formed therein that provides entry into an opening or receptacle 242 defined by the annular-shaped holder 238. At the location of slit 240, the holder 238 includes a pair of protrusions 244 that extend radially outward from a remainder of the holder 238, that provide structure about the slit 240.

The catch clip 236 also includes finger grips 246 positioned on opposing sides of the annular-shaped holder 238 and extending radially outward therefrom, such as at an angle that is less than 90 degrees, e.g., 45 degrees. The finger grips 246 are configured to be engaged by fingers of a user and have a pinching force applied thereto. When a pinching force is applied to the finger grips 246, the catch clip 236 is caused to flex such that a width/size of the slit 240 is increased and access to the opening/receptacle 242 of holder 238 is provided. In this open configuration, the catch clip 236 may be positioned about the engaged port 62 and spike portion 50, with the port 62 and spike portion 50 retained within holder 238.

The annular-shaped holder 238 is formed of an upper ring 248 and a lower ring 250 that are separated by an annular gap 252 therebetween, with the upper ring 248 and lower ring 250 joined together by the finger grips 246 on opposing sides of the holder 238. The annular gap 252 is sized to accommodate positioning of a spike collar 54 and port collar 65 therein (when spike portion 50 is pushed into port 62 and the spike collar 54 and port collar 65 are in contact with each other). Thus, with the catch clip 236 positioned about the engaged port 62 and the spike portion 50, the spike collar 54 and port collar 65 are retained within annular gap 252, such that the catch clip 236 restricts vertical movement between the port 62 and the spike portion 50, so as to prevent dislodgement of the spike portion 50 from the port 62 and ensure that adapter 10 remains connected to infusion fluid container 60.

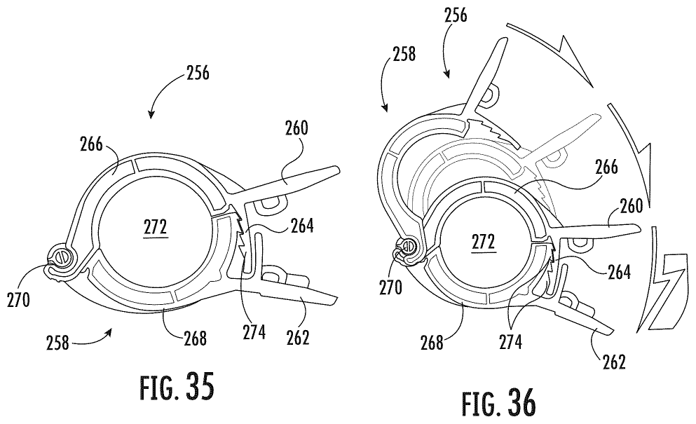

In the embodiment of FIGS. 35 and 36, a catch clip 256 is illustrated that includes an annular-shaped holder 258, first and second actuation arms 260, 262 extending out from the holder 258, and a locking arm 264. The annular-shaped holder 258 is formed of two semi-circular holder portions 266, 268 that are connected together at a pivot point 270 (such as via a pin connection), such that the first portion 266 can be rotated relative to the second portion 268 to provide access to an opening or receptacle 272 defined by the annular-shaped holder 258.

The first and second actuation arms 260, 262 are provided on the first and second holder portions 266, 268, respectively, to provide for relative movement between the first and second holder portions 266, 268. Via first and second actuation arms 260, 262, the catch clip 256 may be reconfigured from an open configuration (where the first holder portion 266 is rotated away from second holder portion 268 and access is provided to the opening/receptacle 272 of the holder 258) to a closed configuration (where the first holder portion 266 is brought into contact with second holder portion 268 and the opening/receptacle 272 of the holder 258 is closed off). In the open configuration, the catch clip 256 may be positioned about the engaged port 62 and spike portion 50, with the port 62 and spike portion 50 retained within holder 258.

The locking arm 264 is joined to the first holder portion 266 (or first actuation arm 260) and is oriented such that it may be brought into contact/engagement with second holder portion 268 when the catch clip 256 is moved to the closed configuration. According to embodiments, locking arm 264 includes a plurality or serrations or teeth 274 formed thereon that may engage serrations or teeth 274 formed on an outer surface of second holder portion 268. When the teeth 274 of locking arm 264 are engaged with teeth 274 on second holder portion 268, the catch clip 256 may be locked in the closed configuration.

While not shown in FIGS. 35 and 36, it is recognized that similar as to the catch clip 256 of FIGS. 33 and 34, the annular-shaped holder 258 (i.e., each of first and second holder portions 266, 268) is formed of an upper member 248 and a lower member 250 that are separated by a gap 252 therebetween. The gap 252 is sized to accommodate positioning of spike collar 54 and port collar 65 therein (when spike portion 50 is pushed into port 62 and the spike collar 54 and port collar 65 are in contact with each other). Thus, with the catch clip 256 positioned about the engaged port 62 and the spike portion 50, the spike collar 54 and port collar 65 are retained within gap 252, such that the catch clip 256 restricts vertical movement between the port 62 and the spike portion 50, so as to prevent dislodgement of the spike portion 50 from the port 62 and ensure that adapter 10 remains connected to infusion fluid container 60.

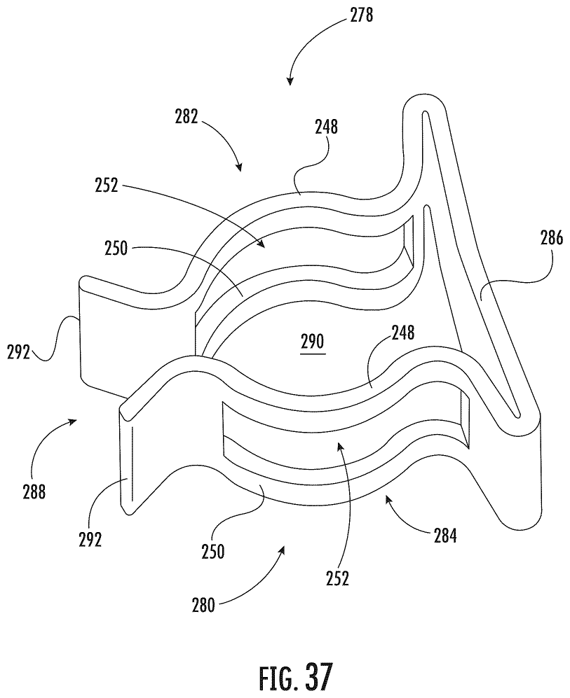

In the embodiment of FIG. 37, a catch clip 278 is illustrated that is formed as a singular member that may be pushed onto the engaged port 62 and spike portion 50. The catch clip 278 includes a holder 280 formed of a first holder arm 282 and a second holder arm 284, and a connecting member 286 that is coupled to each of the first and second holder arms 282, 284 at one end thereof. The first and second holder arms 282, 284 have a generally C-shaped construction, with one end thereof joined to connecting member 286 and an opposing end being free. The free end of the first and second holder arms 282, 284 are separated by a small gap or slit 288, a size of which may be increased to provide entry into an opening or receptacle 290 defined by the C-shaped holder arms 282, 284. At the location of slit 288, each of the holder arms 282, 284 includes a protrusion 292 thereon. The protrusions 292 formed on the holder arms 282, 284 are angled away from each other, so as to provide a funnel or ramp sloping toward the slit 288—with the funnel/ramps allowing for an engaged port 62 and spike portion 50 to be pushed into and through the slit 288, and into the opening/receptacle 290 of the catch clip 278.

Similar as to the catch clips 236, 256 of FIGS. 33-34 and FIGS. 35-36, each of first and second holder arms 282, 284 of catch clip 278 is formed of an upper member 248 and a lower member 250 that are separated by a gap 252 therebetween. The gap 252 is sized to accommodate positioning of a spike collar 54 and port collar 65 therein (when spike portion 50 is pushed into port 62 and the spike collar 54 and port collar 65 are in contact with each other). Thus, with the catch clip 278 positioned about the engaged port 62 and the spike portion 50, the spike collar 54 and port collar 65 are retained within gap 252, such that the catch clip 278 restricts vertical movement between the port 62 and the spike portion 50, so as to prevent dislodgement of the spike portion 50 from the port 62 and ensure that adapter 10 remains connected to infusion fluid container 60.

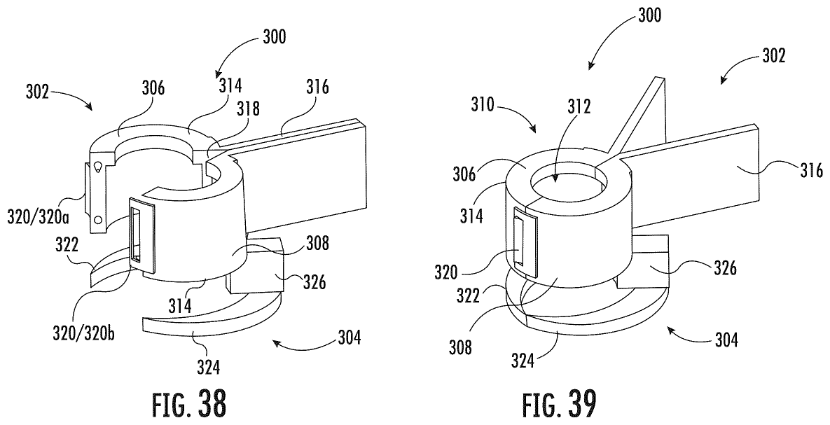

In the embodiment of FIGS. 38-40, a catch clip 300 is illustrated that comprises a clip portion 302 and separate claw portion 304. The clip portion 302 includes a first clip arm 306 and second clip arm 308 that, when in a closed arrangement, provide a holder 310 defining an opening or receptacle 312 within which the engaged port 62 and spike portion 50 may be retained. The first clip arm 306 and second clip arm 308 include a generally C-shaped portion 314, as well as a finger grip portion 316. The first clip arm 306 and second clip arm 308 may be joined together via a spring 318 that is positioned at a location between the C-shaped portion 314 and finger grip portion 316. The spring 318 may bias the first clip arm 306 and second clip arm 308 in the closed arrangement, with the first clip arm 306 and second clip arm 308 moving to an open arrangement upon a pinching force being applied to the finger grip portions 316.

In some embodiments, the catch clip 300 may include a locking mechanism 320 via which the first clip arm 306 and second clip arm 308 of the catch clip 300 may be locked together to secure the catch clip 300 in place about the engaged port 62 and spike portion 50. In an exemplary embodiment, the first clip arm 306 and second clip arm 308 of the clamp may be locked together via a locking mechanism 320 comprising a tongue and groove type lock mechanism. That is, one clip arm 306 of the catch clip 300 may include a tab or tongue 320a formed at the end thereof, while the other clip arm 308 of the catch clip 300 may include a hook or groove 320b formed therein configured to receive the tongue 320a therein. A user may push the tongue 320a into engagement with the hook/groove 320b upon positioning of the catch clip 300 about the port 62 and spike portion 50, to thus lock the catch clip 300 in a closed configuration.

The claw portion 304 comprises a first claw arm 322 and second claw arm 324 each formed as generally C-shaped arm. The first claw arm 322 and second claw arm 324 are joined to the first clip arm 306 and second clip arm 308 by support members 326, with the support members 326 spacing the first claw arm 322 and second claw arm 324 vertically apart from the first clip arm 306 and second clip arm 308. The support members 326 join the first claw arm 322 and second claw arm 324 to the first clip arm 306 and second clip arm 308, respectively, such that movement/actuation of the first clip arm 306 and second clip arm 308 causes a reciprocal movement of the first claw arm 322 and second claw arm 324.

When secured to the engaged port 62 and spike portion 50, the clip arms 306, 308 may engage the port collar 65 (and, optionally, the spike collar 54), so as to secure the catch clip 300 to the infusion fluid container 60. Additionally, the claw arms 322, 324 may engage a portion of the main body 36 of the infusion adapter 10 (e.g., stop 56 of main body 36), so as to secure the catch clip 300 to the infusion adapter 10. Thus, with the catch clip 300 positioned about the engaged port 62 and the spike portion 50, the catch clip 300 restricts vertical movement between the port 62 and the spike portion 50, so as to prevent dislodgement of the spike portion 50 from the port 62 and ensure that adapter 10 remains connected to infusion fluid container 60.

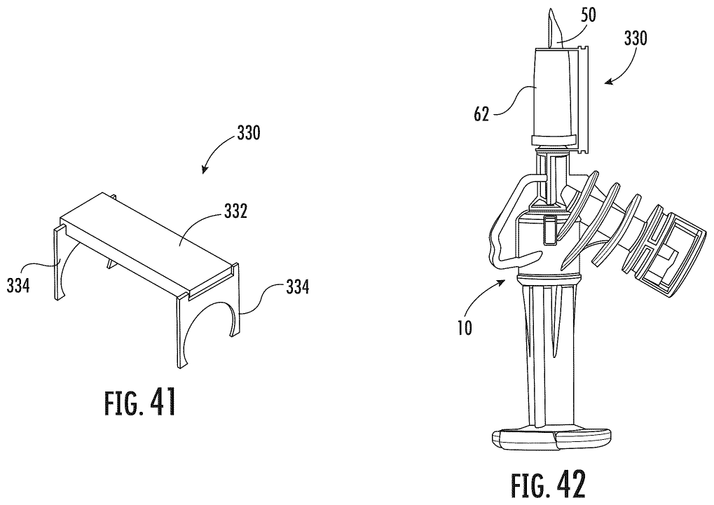

In the embodiment of FIGS. 41 and 42, a catch clip 330 is illustrated that comprises an elongated clip having a flat extension member 332 and a flexible C-shaped clip 334 attached to each end of the extension member 332. The extension member 332 is sized such that a length thereof spaces the C-shaped clips 334 apart by a distance where one of the C-shaped clips 334 is positioned above the port collar 65 and the other C-shaped clip 334 is positioned below the spike collar 54. The C-shaped clips 334 may flex as the catch clip 330 is pushed onto the engaged port 62 and spike portion 50, with the C-shaped clips 334 engaging therewith via an interference fit. Thus, with the catch clip 330 positioned about the engaged port 62 and the spike portion 50 and one C-shaped clip 334 positioned above the port collar 65 and the other C-shaped clip 334 positioned below the spike collar 54, the catch clip 330 restricts vertical movement between the port 62 and the spike portion 50, so as to prevent dislodgement of the spike portion 50 from the port 62 and ensure that adapter 10 remains connected to infusion fluid container 60.

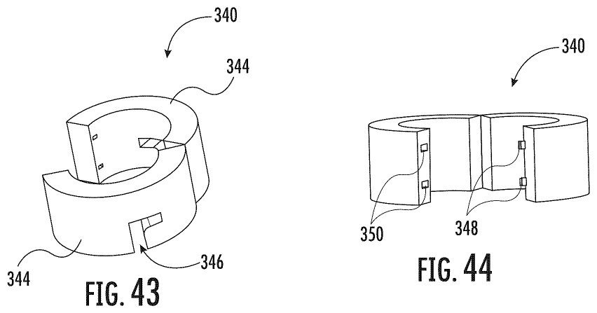

Referring to FIGS. 43-45, a clip-type securing mechanism 340 that may be attached/added after mating of the infusion adapter 10 and infusion fluid container 60 is illustrated, according to an embodiment of the disclosure. The clip-type securing mechanism 340 may be applied onto the port 62 and spike portion 50 after engagement thereof, with the clip-type securing mechanism 340 connecting to stems 342 formed/molded on the adapter 10 (i.e., extending out from stop 56 of main body 36) via a bayonet connection formed in the clip 340, to secure the clip 340 relative to the port 62 and adapter 10. The clip-type securing mechanism 340 is thus referred to here below as a “bayonet clip 340.”

The bayonet clip 340 is formed of two C-shaped clip halves 344 that are joined together via a flexible hinge 346. The C-shaped clip halves 344 may be actuated from an open configuration to a closed configuration, with the bayonet clip 340 actuated to the closed configuration after placement thereof about the engaged port 62 and spike portion 50. In some embodiments, the inner surface of the C-shaped clip halves 344 may be lined with a soft, elastomeric material (not shown) that provides compliance during locking/closing.

Each clip half 344 includes an L-shaped slot 346 formed therein that is configured to receive a stem 342 of adapter 10 therein, with the placement of stems 342 in L-shaped slots 346 functioning to lock the bayonet clip 340 in place once placed about the engaged port 62 and spike portion 50. Each of the clip half 344 also includes thereon a component of a locking mechanism via which the clip halves 344 may be locked together to secure the bayonet clip 340 in place about the engaged port 62 and spike portion 50. In an exemplary embodiment, the C-shaped clip halves 344 may be locked together via engagement of snaps 348 on one clip half 344 with receptacles or slots 350 on the other clip half 344. A user may push the snaps 348 into engagement with the receptacles/slots 350 upon positioning of the bayonet clip 340 about the port 62 and spike portion 50, to thus lock the bayonet clip 340 in a closed configuration.

With the bayonet clip 340 positioned about the engaged port 62 and the spike portion 50, the bayonet clip 340 restricts vertical movement between the port 62 and the spike portion 50, so as to prevent dislodgement of the spike portion 50 from the port 62 and ensure that adapter 10 remains connected to infusion fluid container 60.

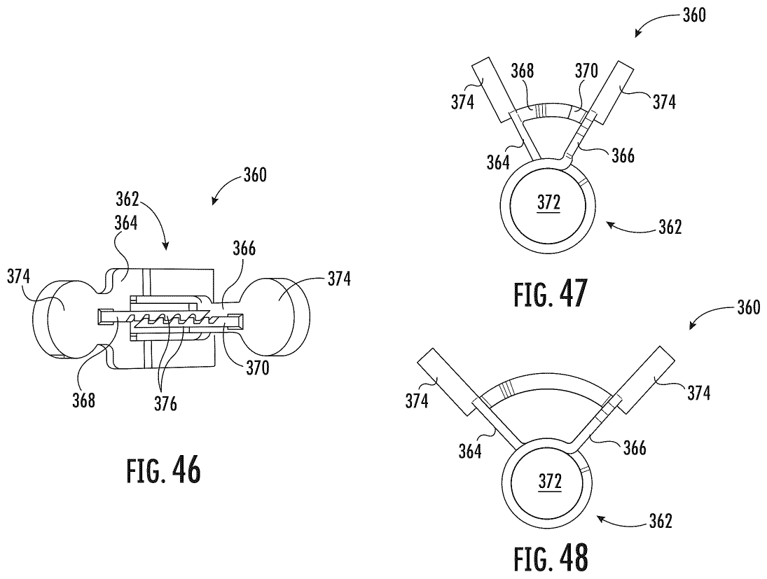

Referring to FIGS. 46-49, a clamp-type securing mechanism 360 (“clamp 360”) is provided that may be applied onto the port 62 or the spike portion 50 prior to engagement therebetween, with the clamp 360 subsequently moved into place and tightened after engagement to secure the port 62 and spike portion 50 together.

In the embodiment of FIGS. 46-49, a clamp 360 is illustrated that is configured as a spring clamp. The spring clamp 360 is formed as a single, integral component that may be characterized as including a holder 362, first and second actuation arms 364, 366, and first and second locking arms 368, 370. The holder 362 defines a circular opening or receptacle 372 sized to receive the port 62 and spike portion 50 therein, with a size of the opening/receptacle 372 being varied based on a positioning of the first and second actuation arms 364, 366. The first and second actuation arms 364, 366 extend out from the holder 362 and are in a crossed arrangement-with the first actuation arm 364 and second actuation arm 366 being vertically offset to allow for crossing of the arms. Each of the first and second actuation arms 364, 366 includes a thumb press 374 at the end thereof via which a user may engage the arms 364, 366, with the user able to apply a pinching force to the thumb presses 374 to cause movement/deflection of the first and second actuation arms 364, 366, thereby altering a size of the opening/receptacle 372 of the holder 362. The first and second locking arms 368, 370 are joined to the first and second actuation arms 364, 366 and are oriented generally orthogonal thereto-with the first and second locking arms 368, 370 extending away from the first and second actuation arms 364, 366, respectively, and toward each other, such that the first and second locking arms 368, 370 may selectively contact/engage each other based on positioning of the first and second actuation arms 364, 366. In some embodiments, each of the first and second locking arms 368, 370 may comprise a curved or arc-shaped arm, to better provide for engagement therebetween. According to embodiments, each of the first and second locking arms 368, 370 includes a plurality or serrations or teeth 376 formed thereon, with one of the locking arms 368, 370 having teeth 376 on a top surface thereof and the other of the locking arms 368, 370 having teeth 376 on a bottom surface thereof, such that the teeth 376 may engage each other When engaged, the first and second locking arms 368, 370 may function similar to a locking forceps, with positioning of the first and second actuation arms 364, 366 and a sizing of the holder 362 being locked in-place, even upon termination of a pressure being applied to thumb presses 374.

In using the spring clamp 360 to secure the infusion adapter 10 to the infusion fluid container 60, the clamp 360 is initially provided and positioned about the port 62 or spike member 50, with the port 62 or spike member 50 positioned within the opening/receptacle 372 of holder 362. The clamp 360 is initially provided in an expanded position, where the opening/receptacle 372 of holder 362 is sized larger than the port 62 or spike member 50, so as to be in no/minimal contact therewith. The clamp 360 is retained in the initial expanded position via engagement of the teeth 376 on the first and second locking arms 368, 370. Upon placement of the spring clamp 360, the spike portion 50 is then pushed into port 62 to engage the adapter 10 with the infusion fluid container 60. Once the spike portion 50 is completely inserted into the port 62, the clamp 360 is actuated by a user-with the user applying a pinching force to the two thumb presses 374 that brings the teeth 376 of the first and second locking arms 368, 370 out of engagement, i.e., the teeth 376 of the first and second locking arms 368, 370 may separate vertically from each other upon a pinching force being applied to thumb presses 374. With the teeth 376 of the first and second locking arms 368, 370 disengaged, the user may lessen the pinching force being applied and allow the spring clamp 360 to transition from the expanded position toward a tightened position-with the first and second actuation arms 364, 366 moving away from each other, which in turn causes the size of the opening/receptacle 372 of holder 362 to decrease. As the size of opening/receptacle 372 is reduced, the holder 362 applies a compression force against the port 62 and the spike portion 50 positioned therein that prevents dislodgement of the spike portion 50 from the port 62, such that adapter 10 remains connected to infusion fluid container 60. The holder 362 may be retained or locked in the closed position via re-engaging of the teeth 376 of the first and second locking arms 368, 370.

Beneficially, embodiments of the invention thus are directed to securing mechanisms for securely engaging an infusion adapter with an infusion fluid container, in order to prevent dislodging of the spike connector of the adapter from the injection port and potential medication leakage from the infusion fluid container. The securing mechanism is positionable about the injection port of the infusion fluid container and the main spike portion of the infusion adapter after the main spike portion has been engaged with the injection port. The securing mechanism functions to secure the injection port and the main spike portion together by restricting vertical movement between the port and the main spike portion, so as to prevent dislodging of the main spike portion from the injection port.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A fluid transfer system comprising:
an infusion fluid container containing an infusion fluid therein, the infusion fluid container comprising a port having a fluid barrier member therein;
an infusion adapter for connection with the infusion fluid container, the infusion adapter comprising a connection spike positioned at a first end of the infusion adapter, the connection spike having a main spike portion and a puncturing point configured to engage the port, with the port positioned about the main spike portion and the puncturing point when engaged therewith; and
a securing mechanism positionable about the port and the main spike portion with the main spike portion engaged with the port, to secure the port and the main spike portion together;
wherein the securing mechanism restricts vertical movement between the port and the main spike portion to prevent dislodging of the main spike portion from the port.

2. The fluid transfer system of claim 1, wherein the securing mechanism comprises a hinge clamp configured to apply a compression force against the engaged port and main spike portion to prevent dislodging of the main spike portion from the port.

3. The fluid transfer system of claim 2, wherein the hinge clamp comprises:
a clamp member including a flexible leg assembly and an annular-shaped holder with slits formed therein to separate the holder into a pair of holder portions that may be selectively joined and separated, the holder positionable about the engaged port and main spike portion and configured to apply the compression force thereto; and
a slider translatable relative to the clamp member, with the slider applying an inward-directed force against the flexible leg assembly as the slider is moved from a first end of the flexible leg assembly toward a second end of the flexible leg assembly, thereby causing the pair of holder portions to join and apply the compression force to the engaged port and main spike portion.

4. The fluid transfer system of claim 3, wherein the flexible leg assembly comprises a first leg and a second leg each including a plurality of teeth formed on an outward facing surface thereof, and wherein the slider comprises a pair of tongues that engage with the plurality of teeth on the first and second legs, with the pair of tongues engaging with the plurality of teeth to prevent movement of the slider from the second end of the flexible leg assembly back toward the first end of the flexible leg assembly, so as to lock the annular-shaped holder in a closed configuration.

5. The fluid transfer system of claim 3, wherein the hinge clamp further comprises a screw adjuster operable to actuate the slider, the screw adjuster including:
a thumb screw inserted through a hole in the flexible leg assembly, with the thumb screw being rotatable to linearly advance the thumb screw relative to the flexible leg assembly; and
a slider connector coupling the thumb screw to the slider, such that linear movement of the thumb screw relative to the clamp member is transferred to slider, with the slider thus moving linearly relative to the clamp member responsive to rotation of the thumb screw.

6. The fluid transfer system of claim 2 wherein the hinge clamp comprises a bi-stable hinge clamp including:
a clamp member including a first leg and a second leg joined together at a first end thereof via a connecting portion;
an annular-shaped holder with slits formed therein to separate the holder into a pair of holder portions, with a respective holder portion joined to each of the first leg and the second leg;
a shaft inserted through a hole in the connecting portion so as to be movable relative to the clamp member, the shaft including a button at a first end thereof; and
a live hinge connecting the clamp member to the shaft, the live hinge comprising a plurality of hinge members that extend between the shaft and each of the first leg and the second leg, wherein the plurality of hinge members comprise flexible members that are actuatable from an extended configuration to a collapsed configuration responsive to movement of the shaft;
wherein with the shaft in a first position, the hinge members are in the extended configuration, so that the first leg and the second leg are in an open position, with the pair of holder portions thus separated from one another; and
wherein with the shaft in a second position, the hinge members are in the collapsed configuration, so that the first leg and the second leg are in a closed position, with the pair of holder portions moved into contact with each other to apply the compression force to the engaged port and main spike portion.

7. The fluid transfer system of claim 1, wherein the securing mechanism comprises a sleeve clamp including a pair of flexible sleeve portions separated by a slit, with the slit being expandable upon a pressure being applied thereto, so as to enable the engaged port and spike portion to be pushed into a receptacle defined by the pair of flexible sleeve portions.

8. The fluid transfer system of claim 7, wherein the sleeve clamp comprises a locking mechanism configured to lock the pair of flexible sleeve portions together.

9. The fluid transfer system of claim 7, wherein an inner surface of the pair of flexible sleeve portions comprises a plurality of teeth spaced circumferentially thereabout, with each of the plurality of teeth extending radially inward and providing increased friction between the sleeve clamp and the port.

10. The fluid transfer system of claim 1, wherein the connection spike comprises a spike collar formed about the main spike portion on an end thereof opposite the puncturing point, and/or wherein the port comprises a port collar formed thereon.

11. The fluid transfer system of claim 10, wherein the securing mechanism comprises a hinge lock enclosure clamp including:
a pair of brackets coupled together in a hinged arrangement, with the hinge lock enclosure clamp enclosing a region where the port is engaged to the spike portion; and
a locking mechanism provided on the pair of brackets and configured to lock the pair of brackets together.

12. The fluid transfer system of claim 11, wherein each of the pair of brackets comprises a C-shaped channel defined therein that extends a length of the bracket, with opposing end walls of the bracket having semi-circular cut-out formed therein and a pair of C-shaped protrusions being provided on an inner surface of the bracket

13. The fluid transfer system of claim 12, wherein with the hinge lock enclosure clamp positioned about the engaged port and main spike portion, the spike collar and the port collar are retained between the pair of C-shaped protrusions, so as to restrict vertical movement between the port and the main spike portion.

14. The fluid transfer system of claim 11, wherein each of the pair of C-shaped protrusions comprises a silicone pad thereon that forms a seal with the port and the main spike portion, to prevent leakage therefrom.

15. The fluid transfer system of claim 11, wherein at least one of the pair of brackets comprises a rubber gasket arranged about a perimeter of the bracket, where the bracket is brought into engagement with the other bracket, with the rubber gasket seals an interior of the hinge lock enclosure clamp to prevent any leakage out therefrom.

16. The fluid transfer system of claim 15, wherein the hinge lock enclosure clamp comprises an absorbing material included within the interior that is configured to absorb any leakage of fluid.

17. The fluid transfer system of claim 15, wherein each of the pair of brackets comprises a C-shaped channel defined therein that extends a length of the bracket, with opposing end walls of the bracket having semi-circular cut-out formed therein, and wherein each of the pair of brackets comprises a C-shaped slot formed on an inner surface thereof configured to receive and retain the spike collar therein, and thereby restrict vertical movement between the port and the main spike portion.

18. The fluid transfer system of claim 17, wherein these semi-circular cut-out includes an adhesive applied thereto that is configured to secure the hinge lock enclosure clamp to the insertion port.

19. The fluid transfer system of claim 11, wherein the locking mechanism comprises one of:
a hinge pin on a first bracket of the pair of brackets and a second bracket of the pair of brackets; or
a snap or tab on each of the first bracket and second bracket that are configured to engage each other.

20. The fluid transfer system of claim 10, wherein the securing mechanism comprises a press-fit clamp including a first enclosure tab and a second enclosure tab that, when joined together, define a channel therein configured to receive the engaged port and spike portion, with the first enclosure tab including a plurality of pins thereon configured to engage a plurality of receptacles on the second enclosure tab to lock the press-fit clamp together.

21. The fluid transfer system of claim 20, wherein each of the first and second enclosure tabs comprises a semi-circular cut-out formed at each of opposing ends thereof, with one or more of the semi-circular cut-outs including a rib formed thereabout configured to engage an outer surface of the port when the press-fit clamp is positioned about the engaged port and spike portion, to increase friction between the press-fit clamp and the port.

22. The fluid transfer system of claim 10, wherein the securing mechanism comprises a catch clip that includes an holder that defines an opening or receptacle configured to receive the engaged port and spike portion, the holder comprising an upper member and a lower member separated vertically by a gap, with the gap sized to receive the spike collar and port collar therein, and thereby restrict vertical movement between the port and the main spike portion.

23. The fluid transfer system of claim 22, wherein the holder comprises an annular-shaped holder having a slit formed therein that provides entry into the opening or receptacle, and wherein the catch clip comprises finger grips positioned on opposing sides of the annular-shaped holder and extending radially outward therefrom; and
wherein, responsive to application of a pinching force to the finger grips, the catch clip is caused to flex such that a width of the slit is increased and access to the opening or receptacle of holder is provided.

24. The fluid transfer system of claim 22, wherein the catch clip comprises:
semi-circular first and second holder portions that collectively form the holder, the first and second holder portions connected together at a pivot point and movable between an open position and closed position;
first and second actuation arms provided on the first and second holder portions and actuatable by a user to move the first and second holder portions from the open position to the closed position; and
a locking arm joined to the first holder portion and oriented such that it may be brought into engagement with the second holder portion when the catch clip is moved to the closed position;
wherein the locking arm includes a plurality or serrations or teeth formed thereon that may engage serrations or teeth formed on an outer surface of the second holder portion to lock the catch clip may be locked in the closed position.

25. The fluid transfer system of claim 22, wherein the catch clip comprises:
a first holder arm and a second holder arm that collectively form the holder, each of the first and second holder arms having a C-shaped construction and including a first end and a second end; and
a connecting member coupled to the first and second holder arms at the first end;
wherein the second end of the first holder arm is separated from the second end of the second holder arm by a gap; and
wherein the second end of the first holder arm and the second holder arm each include a protrusions formed thereon, the protrusions on the first and second holder arms angled away from each other, so as to provide a funnel sloping toward the gap, with a size of the gap expanding upon a pushing of the catch clip onto the engaged port and main spike portion.

26. The fluid transfer system of claim 10, wherein the securing mechanism comprises a catch clip including:
a clip portion comprising:
a first clip arm and a second clip arm that, when in a closed arrangement, provide a holder defining an opening or receptacle within which the engaged port and spike portion may be retained, each of the first clip arm and a second clip arm including a C-shaped portion and a finger grip portion;
a spring coupling the first clip arm and the second clip arm and that biases the first clip arm and second clip arm in the closed arrangement, with the first clip arm and second clip arm moving to an open arrangement upon a pinching force being applied to the finger grip portions; and
a locking mechanism via which the first clip arm and second clip arm may be locked together to secure the catch clamp in place about the engaged port and spike portion; and
a claw portion comprising a first claw arm and a second claw arm each formed as a C-shaped arm, the first claw arm and the second claw arm joined to the first clip arm and the second clip arm, respectively, by support members, with movement of the first clip arm and the second clip arm causing a corresponding movement of the first claw arm and the second claw arm;
wherein the support members space the claw arm and the second claw arm vertically apart from the first clip arm and the second clip arm, such that the first and second clip arms engage the port collar and the spike collar and such that the claw arms engage a main body of the infusion adapter.

27. The fluid transfer system of claim 10, wherein the securing mechanism comprises a catch clip including:
a flat extension member having a first end and a second end;
a first C-shaped clip affixed to the first end of the flat extension member; and
a second C-shaped clip affixed to the second end of the flat extension member;
wherein the first and second C-shaped clips are spaced apart by the flat extension member such that, when the catch clip is engaged with the port and the main spike portion, the first C-shaped clip is positioned above the port collar and the second C-shaped clip is positioned below the spike collar; and
wherein the first and second C-shaped clips comprise flexible clips configured to engage the port and the main spike portion via an interference fit.

28. The fluid transfer system of claim 1, wherein the infusion adapter comprises a pair of stems positioned at the first end of the infusion adapter and on opposing sides of the connection spike; and
wherein the securing mechanism comprises a bayonet clip including a pair of C-shaped clip halves joined together via a flexible hinge, so as to be movable from an open position to a closed position, each of the pair of C-shaped clip halves including an L-shaped slot formed therein configured to receive a respective one of the pair of stems therein;
wherein with the bayonet clip positioned about the engaged port and spike portion, the bayonet clip is locked in place relative to the infusion adapter via engagement of the stems with the L-shaped slots.

29. The fluid transfer system of claim 28, wherein the bayonet clip further comprises a locking mechanism via which the C-shaped clip halves may be locked together to secure the bayonet clip in the closed position about the engaged port and spike portion.

30. The fluid transfer system of claim 1, wherein the securing mechanism comprises a spring clamp including:
an annular holder defining an opening sized to receive the engaged port and spike portion therein;
first and second actuation arms extending out from the annular holder in a crossed arrangement, with the first actuation arm and the second actuation arm being vertically offset to allow for crossing of the first and second actuation arms, wherein each of the first and second actuation arms includes a thumb press via which the first and second actuation arms may be actuated; and
first and second locking arms joined to the first and second actuation arms, respectively, with the first and second locking arm extending toward each other, such that the first and second locking arms may selectively engage each other based on positioning of the first and second actuation arms, wherein each of the first and second locking arms includes a plurality or teeth formed thereon, with the first locking arm having teeth on a top surface thereof and the second locking arm having teeth on a bottom surface thereof, such that the teeth may engage each other.

31. The fluid transfer system of claim 30, wherein the annular holder is retained in an initial expanded position via engagement of the teeth on the first and second locking arms, and wherein upon application of a pinching force to the thumb presses, the teeth of the first and second locking arms are brought out of engagement, allowing the first and second actuation arms to separate further, and moving the annular holder from the expanded position to a tightened position, where the spring clamp applies a compression force against the engaged port and main spike portion to prevent dislodging of the main spike portion from the port.
